(19) United States
(12) Patent Application Publication
(10) Pub. No.: US 2023/0233062 A1
(43) Pub. Date: Jul. 27, 2023

(54) ENDOSCOPE

(71) Applicant: FUJIFILM Corporation, Tokyo (JP)

(72) Inventor: Takashi HARADA, Kanagawa (JP)

(73) Assignee: FUJIFILM Corporation, Tokyo (JP)

(21) Appl. No.: 18/191,865

(22) Filed: Mar. 28, 2023

Related U.S. Application Data
(63) Continuation of application No. PCT/JP2021/036390, filed on Oct. 1, 2021.

(30) Foreign Application Priority Data
Oct. 2, 2020 (JP) 2020-167396

(51) Int. Cl. A61B 1/00; A61B 1/018

(52) U.S. Cl. CPC A61B 1/00098; A61B 1/018

(57) ABSTRACT

A wire fixation mechanism (78) includes a wire catch (100), a catch guide (102), and a sliding lever (80), a wire (38) includes a locking target portion (39) at a proximal end thereof, and the wire catch (100) includes a locking hole (137) into which the locking target portion (39) is insertable and at which the locking target portion (39) is lockable and a fixing member (106) that fixes a locked state between the locking target portion (39) and the locking hole (137).

### CROSS-REFERENCE TO RELATED APPLICATIONS

The present application is a Continuation of PCT International Application No. PCT/JP2021/036390 filed on Oct. 1, 2021 claiming priority under 35 U.S.C § 119(a) to Japanese Patent Application No. 2020-167396 filed on Oct. 2, 2020. Each of the above applications is hereby expressly incorporated by reference, in its entirety, into the present application.

### BACKGROUND OF THE INVENTION

### 1. Field of the Invention

The present invention relates to an endoscope, and particularly relates to an endoscope in which an elevator that changes the direction of extraction of a treatment tool is provided on a distal end side of an insertion portion.

### 2. Description of the Related Art

Regarding an endoscope, various treatment tools are introduced through a treatment tool inlet port provided in an operation portion and the treatment tools are used for treatment after being extracted to the outside through a treatment tool outlet port open at a distal end portion of the insertion portion. For example, in the case of a duodenoscope, a treatment tool such as a guide wire or a contrast tube is used. In the case of an ultrasonic endoscope, a treatment tool such as a puncture needle is used. In the case of a front-viewing endoscope and an oblique-viewing endoscope which are endoscopes other than the endoscopes described above, a treatment tool such as forceps or a snare is used. The direction of extraction of such a treatment tool needs to be changed at the distal end portion so that treatment is performed on a desired position in a subject. Therefore, a distal end portion main body of the distal end portion is provided with an elevator that changes the direction of extraction of the treatment tool. The endoscope is provided with a treatment tool elevation mechanism that changes the posture of the elevator between an elevation position and a laid-down position.

An endoscope disclosed in US2007/0099500A includes an elevator provided on a distal end side of a working channel. The elevator is rotated about a pivot shaft by a wire and thus a medical instrument can be accurately directed to a surgical site. A proximal end side of the wire is inserted into a collet, and then a nut is rotated so that the collet is tightened and the wire is fixed by the collet.

### SUMMARY OF THE INVENTION

However, in the case of the endoscope disclosed in US2007/0099500A, the collet and the nut are configured as separate components, and the wire is fixed by a plurality of members. Therefore, it is necessary to insert the wire into the collet and push the collet into the nut for rotation. Therefore, an operation for fixation of the wire may become complicated.

In addition, in the case of a collet chuck, a fixation force may not be sufficiently exerted depending on the degree of fastening, or a wire fixation end may be gripped by a collet at a halfway position (for example, in a state where there is almost no gripping margin of the wire fixation end). Therefore, in a case where a wire is fixed in the above-described state, the wire may fall off from a collet in the case of an elevation operation.

The present invention has been made in consideration of such circumstances and an object of the present invention is to provide an endoscope with which it is possible to reliably fix an elevation operation wire to a wire fixation mechanism.

In order to solve the above-described problems, an endoscope according to an aspect of the present invention comprises an operation portion that is provided with an operation member, an insertion portion that is provided on a distal end side of the operation portion and that is inserted into a subject, a treatment tool elevator that is provided at a distal end portion of the insertion portion, an elevation operation wire of which a distal end side is connected to the treatment tool elevator and that is pushed and pulled, as the operation member is operated, so that the treatment tool elevator is operated, and a wire fixation mechanism that fixes a proximal end side of the elevation operation wire. The wire fixation mechanism includes a wire catch that attachably and detachably locks and fixes the proximal end side of the elevation operation wire, a catch guide that guides the wire catch in a wire axis direction of the elevation operation wire, and a sliding lever that operates as the operation member is operated so that the wire catch is moved forward and backward in the wire axis direction, the elevation operation wire includes a locking target portion that is positioned on a proximal end side of a long wire main body and is formed to have an outer shape larger than an outer shape of the wire main body, and the wire catch includes a locking hole into which the locking target portion is insertable and at which the locking target portion is lockable, and a fixation portion that fixes a locked state between the locking target portion inserted into the locking hole and the locking hole.

According to an aspect of the present invention, it is preferable that the fixation portion includes a restriction surface that restricts the locking target portion locked at the locking hole from moving in a direction orthogonal to the wire axis direction.

According to an aspect of the present invention, it is preferable that the fixation portion includes a fixation hole in which an opening portion, in which the locking target portion is accommodable, is formed and at least a portion of an inner wall surface of the fixation hole is configured as the restriction surface.

According to an aspect of the present invention, it is preferable that the fixation hole includes a conical guide surface that becomes narrower toward an inner portion of the fixation hole.

According to an aspect of the present invention, it is preferable that the locking hole has an opening shape in which a first hole of which a size is enough for the locking target portion to be inserted thereinto and a second hole of which an outer shape is larger than an outer shape of the wire main body and is smaller than an outer shape of the locking target portion are continuously connected to each other.

According to an aspect of the present invention, it is preferable that the wire catch is configured to be rotatable around a rotation axis eccentric from the elevation operation wire, the locking hole is provided at a position eccentric from the rotation axis, and the first hole and the second hole are formed to be continuously connected to each other along a trajectory of rotation around the rotation axis.

According to an aspect of the present invention, it is preferable that the fixation portion is movable between a fixing position at which the locked state of the locking target portion and the locking hole is fixed and an unfixing position at which the locked state of the locking target portion and the locking hole is unfixed.

According to an aspect of the present invention, it is preferable that the operation portion includes a link member that is operated as the operation member is operated, the sliding lever includes a lever connecting portion that is attachably and detachably connectable to the link member, and the fixation portion is movable between the fixing position and the unfixing position as a lever connection operation of connecting the lever connecting portion to the link member is performed.

According to an aspect of the present invention, it is preferable that the operation portion includes a link member that is operated as the operation member is operated, the sliding lever includes a lever connecting portion that is attachably and detachably connectable to the link member, and the wire fixation mechanism includes an operation restriction portion that is selectively switchable between a restriction state in which a lever connection operation of connecting the lever connecting portion to the link member is restricted in a case where the fixation portion is at the unfixing position and an allowance state in which the lever connection operation is allowed in a case where the fixation portion is at the fixing position.

According to an aspect of the present invention, it is preferable that the wire catch includes a locking member that includes a locking hole and the locking member is movable between an insertion position at which the locking target portion is insertable into the locking hole and a locking position at which the locking target portion is locked at the locking hole.

According to the aspects of the present invention, it is possible to reliably fix an elevation operation wire to a wire fixation mechanism.

### BRIEF DESCRIPTION OF THE DRAWINGS

FIG. 1 is a configuration diagram of an endoscope system including an endoscope.

FIG. 2 is an assembly perspective view of a distal end portion of the endoscope shown in FIG. 1.

FIG. 3 is an enlarged perspective view showing a proximal end portion of an operation portion main body.

FIG. 4 is an enlarged perspective view showing the proximal end portion of the operation portion main body.

FIG. 5 is an explanatory view showing the way in which a first embodiment of a wire fixation mechanism is mounted onto the operation portion main body.

FIG. 6 is an explanatory view showing the way in which the first embodiment of the wire fixation mechanism is mounted onto the operation portion main body.

FIG. 7 is an explanatory view showing the way in which the first embodiment of the wire fixation mechanism is mounted onto the operation portion main body.

FIG. 8 is an explanatory view showing the way in which a wire fixation mechanism of the first embodiment is mounted onto the operation portion main body.

FIG. 9 is an explanatory view of connection of a sliding lever to an elevation operation lever.

FIG. 10 is a front view of the operation portion main body with the sliding lever connected to the elevation operation lever.

FIG. 11 is a front view of the operation portion main body with the elevation operation lever positioned at an elevation operation position.

FIG. 12 is a front view of the wire fixation mechanism.

FIG. 13 is a perspective view of a main part in the case of removal of a cap from the wire fixation mechanism shown in FIG. 12.

FIG. 14 is a perspective view of a main part showing a configuration of the sliding lever.

FIG. 15 is a perspective view showing the way in which the wire fixation mechanism shown in FIG. 13 is mounted onto the operation portion main body.

FIG. 16 is a cross-sectional view of the wire fixation mechanism which is taken along line XVI-XVI line of FIG. 15.

FIG. 17 is an explanatory view showing a state where a locking target portion protrudes from a locking hole.

FIG. 18 is an explanatory view showing a state where a wire protrudes from the locking hole.

FIG. 19 is an explanatory view showing a state where the wire is locked at a second hole.

FIG. 20 is a front view of a wire catch in a state as shown in FIG. 19.

FIG. 21 is a front view showing a state where the wire fixation mechanism is mounted onto the operation portion main body.

FIG. 22 is a front view showing a state where a lever connection operation is started.

FIG. 23 is an explanatory view showing a state where the locking target portion is engaged with a recess portion during the lever connection operation.

FIG. 24 is a front view showing a state where the first half of the lever connection operation is finished.

FIG. 25 is a cross-sectional view of the wire fixation mechanism in a state as shown in FIG. 24.

FIG. 26 is a front view showing a state where the second half of the lever connection operation is started.

FIG. 27 is a front view showing a state where the lever connection operation is finished.

FIG. 28 is an explanatory view showing a wire fixation range and a drive range.

FIG. 29 is a perspective view showing a wire fixation mechanism according to a second embodiment.

FIG. 30 is a front view of the wire fixation mechanism shown in FIG. 29.

FIG. 31 is a cross-sectional view of the wire fixation mechanism shown in FIG. 30.

FIG. 32 is a front view of the wire fixation mechanism with fixing member moved in a Y (+) direction.

FIG. 33 is a cross-sectional view of the wire fixation mechanism shown in FIG. 32.

FIG. 34 is a front view of the wire fixation mechanism in the case of the start of the lever connection operation.

FIG. 35 is a front view of the wire fixation mechanism with second shafts abutting restriction surfaces.

FIG. 36 is a cross-sectional view showing a main part of a wire fixation mechanism according to a third embodiment.

FIG. 37 is a perspective view of a wire fixation mechanism according to a fourth embodiment.

FIG. 38 is a perspective view of a wire fixation mechanism according to a fifth embodiment.

FIG. 39 is a cross-sectional view of the wire fixation mechanism shown in FIG. 38.

FIG. 40 is a perspective view of a wire fixation mechanism according to a sixth embodiment.

FIG. 41 is a cross-sectional view of the wire fixation mechanism shown in FIG. 40.

FIG. 42 is an assembly perspective view of a wire fixation mechanism according to a seventh embodiment.

FIG. 43 is an assembly perspective view of a wire fixation mechanism according to an eighth embodiment.

FIG. 44 is an assembly perspective view of a wire fixation mechanism according to a ninth embodiment.

FIG. 45 is a cross-sectional view of the wire fixation mechanism shown in FIG. 44.

### DESCRIPTION OF THE PREFERRED EMBODIMENTS

Hereinafter, preferred embodiments of an endoscope according to an embodiment of the present invention will be described with reference to the accompanying drawings.

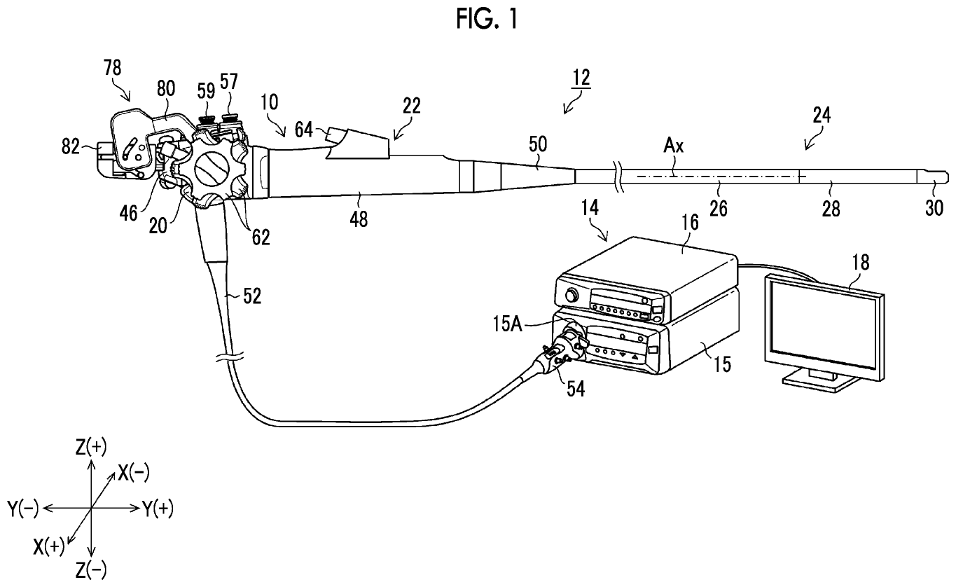

FIG. 1 is a configuration diagram of an endoscope system 12 that includes an endoscope 10 according to an embodiment of the present invention. The endoscope system 12 includes the endoscope 10, an endoscope processor device 14, and a display 18.

The endoscope 10 includes a hand-side operation portion 22 that is provided with an elevation operation lever 20 and an insertion portion 24 that is provided on a distal end side of the hand-side operation portion 22 and is inserted into a subject. The hand-side operation portion 22 functions as an operation portion according to the embodiment of the present invention.

The insertion portion 24 has a major axis direction Ax from a proximal end portion to a distal end portion and includes a soft portion 26, a bendable portion 28, and a distal end portion 30 arranged in order from a proximal end side to a distal end side. A detailed configuration of the distal end portion 30 will be described later. First, a schematic configuration of the distal end portion 30 will be described.

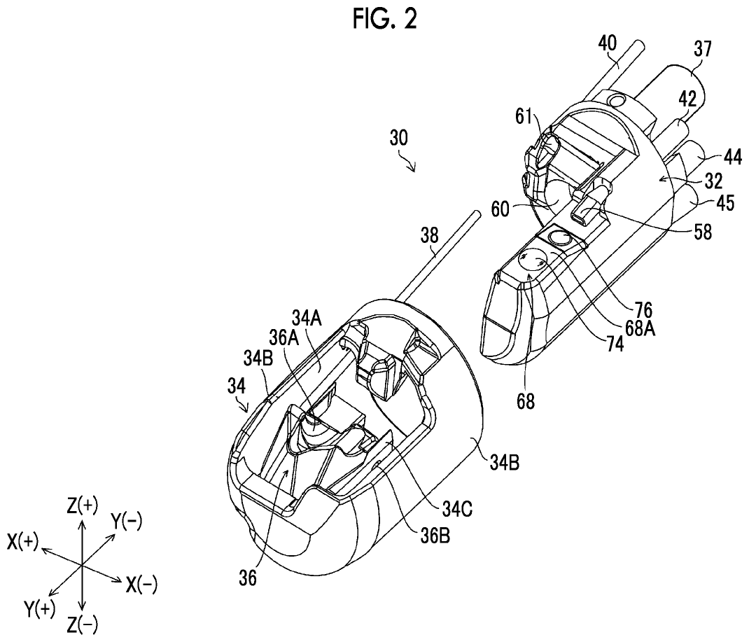

FIG. 2 is an enlarged assembly perspective view of the distal end portion 30. Here, the endoscope 10 of the embodiment (refer to FIG. 1) is a side-viewing endoscope used as, for example, a duodenoscope, and the distal end portion 30 of FIG. 2 has a configuration of a side-viewing endoscope.

As shown in FIG. 2, the distal end portion 30 is configured by mounting a cap 34 to a distal end portion main body 32. The cap 34 is provided with a treatment tool elevator 36 (hereinafter, will be referred to as an elevator 36) including a treatment tool guide surface 36A, and a state in which the elevator 36 is positioned at a laid-down position is shown.

FIG. 2 shows, in addition to the distal end portion 30, various contents disposed inside the insertion portion 24 of the endoscope 10 (refer to FIG. 1). That is, in FIG. 2, a treatment tool channel 37 through which a distal end portion of a treatment tool (not shown) is guided to the distal end portion main body 32, an elevation operation wire 38 (hereinafter, will be referred to as a wire 38) for an operation of changing the direction of extraction of the distal end portion of the treatment tool extracted from the distal end portion main body 32, a wire channel 40 that is composed of a closely attached spring and into which the wire 38 is inserted, an air/water supply tube 42, and a cable insertion channel 44 are shown. In addition, contents such as an insertion channel 45 of a light guide that guides, to the distal end portion main body 32, illumination light supplied from a light source device 15 (refer to FIG. 1) and an angle wire (not shown) for an operation of bending the bendable portion 28 (refer to FIG. 1) are also disposed inside the insertion portion 24.

Note that, in the present specification, the description will be made by using a three-dimensional orthogonal coordinate system of three axis directions (an X-axis direction, a Y-axis direction, and a Z-axis direction). That is, an upward direction will be referred to as a Z (+) direction and a downward direction, which is a direction opposite to the upward direction, will be referred to as a Z (−) direction, where the upward direction is a direction in which the treatment tool (not shown) is extracted by the elevator 36 as seen in a direction from the hand-side operation portion 22 to the distal end portion 30. In addition, a rightward direction at that time will be referred to as an X (+) direction and a leftward direction will be referred to as an X (−) direction. In addition, a frontward direction (a direction to the distal end side in a direction along the major axis direction Ax of the insertion portion 24) at that time will be referred to as a Y (+) direction, and a rearward direction (a direction to a proximal end side in the direction along the major axis direction Ax of the insertion portion 24) will be referred to as a Y (−) direction. Note that, the Y-axis direction including the Y (+) direction and the Y (−) direction is parallel to the direction along the major axis direction Ax of the insertion portion 24 and a wire axis direction of the wire 38. In addition, the Y (+) direction refers to a direction to a distal end side in the wire axis direction, and the Y (−) direction refers to a direction to a proximal end side in the wire axis direction. In addition, the Z-axis direction is a direction orthogonal to the major axis direction Ax, and the X-axis direction is a direction orthogonal to each of the Y-axis direction and the Z-axis direction.

Referring again to FIG. 1, the hand-side operation portion 22 is configured to have a substantially cylindrical shape as a whole. The hand-side operation portion 22 includes an operation portion main body 46 provided with the elevation operation lever 20 and a grip portion 48 connected to the operation portion main body 46. The grip portion 48 is a portion that is gripped by an operator in a case where the endoscope 10 is to be operated, and the proximal end portion of the insertion portion 24 is connected to a distal end side of the grip portion 48 via a bend-proof tube 50.

A proximal end portion of a universal cable 52 is connected to the operation portion main body 46, and a connector device 54 is provided at a distal end portion of the universal cable 52. The connector device 54 is connected to the endoscope processor device 14.

The endoscope processor device 14 includes the light source device 15 and an image processing device 16. The light source device 15 includes a processor-side connector 15A to which the connector device 54 is connected. In addition, the display 18 that displays an image processed by the image processing device 16 is connected to the image processing device 16. The endoscope system 12 has a configuration in which power, optical signals, and the like are transmitted in a contactless manner between the endoscope 10 and the endoscope processor device 14 via a connector unit composed of the connector device 54 and the processor-side connector 15A. Accordingly, light from the light source device 15 is transmitted through an optical fiber cable (not shown) and is emitted from an illumination window 74 (refer to FIG. 2) provided at a distal end surface of the distal end portion 30. In addition, an optical signal, which is obtained by imaging, by means of an imaging element, light captured through an observation window 76 (refer to FIG. 2) and converting the light, is subjected to image processing performed by the image processing device 16 and is displayed by the display 18 in the form of an image.

An air/water supply button 57 and a suction button 59 are arranged to be parallel on the operation portion main body 46. The air/water supply button 57 is a button that can be operated in two stages. Air can be supplied to an air/water supply nozzle 58 (refer to FIG. 2) via the air/water supply tube 42 by means of a first-stage operation and water can be supplied to the air/water supply nozzle 58 via the air/water supply tube 42 by means of a second-stage operation. In addition, it is possible to suck a body fluid such as blood via the treatment tool channel 37 from a treatment tool outlet port 60 (FIG. 2) by operating the suction button 59.

A pair of angle knobs 62 and 62 for an operation of bending the bendable portion 28 is disposed at the operation portion main body 46. The pair of angle knobs 62 and 62 is provided coaxially and rotatably. For example, four angle wires (not shown) are connected to the angle knobs 62 and 62 and the bendable portion 28, and in a case where the angle knobs 62 and 62 are rotated, the angle wires are pushed and pulled and the bendable portion 28 is bent upward, downward, left, and right.

The elevation operation lever 20 is rotatably provided on the operation portion main body 46 to be coaxial with the angle knobs 62 and 62. The elevation operation lever 20 is rotated by a hand of the operator gripping the grip portion 48. The elevation operation lever 20 functions as an operation member according to the embodiment of the present invention.

A wire fixation mechanism 78, which is a first embodiment of a wire fixation mechanism according to the embodiment of the present invention, is provided outside the operation portion main body 46. The wire fixation mechanism 78 includes a sliding lever 80 and a fixation unit 82, and has a configuration for fixation of a proximal end side of the wire 38 (refer to FIG. 2) as described later. One end of the sliding lever 80 is attachably and detachably connected to the elevation operation lever 20 side, and moves (slides) as the elevation operation lever 20 is rotated. In addition, the other end of the sliding lever 80 is provided with the above-described fixation unit 82. The fixation unit 82 is mounted on the operation portion main body 46, and the proximal end side of the wire 38 is fixed to the fixation unit 82. Accordingly, the elevation operation lever 20 and the wire 38 are connected to each other via the wire fixation mechanism 78. Note that the wire fixation mechanism 78 will be described later.

As shown in FIG. 1, the grip portion 48 of the hand-side operation portion 22 includes a treatment tool inlet port 64 into which the treatment tool is introduced. The treatment tool (not shown) that is introduced through the treatment tool inlet port 64 with a distal end portion being at the front is inserted into the treatment tool channel 37 shown in FIG. 2 and is extracted to the outside through the treatment tool outlet port 60. Examples of the treatment tool include a treatment tool such as biopsy forceps of which a distal end portion includes a cup capable of collecting biological tissue, a knife for endoscopic sphincterotomy (EST), and a contrast tube.

Next, the structure of the distal end portion 30 shown in FIG. 2 will be described.

First, the distal end portion main body 32 will be described.

The distal end portion main body 32 is formed of, for example, a corrosion-resistant metal material and includes a partition wall 68 protruding in the Y (+) direction. In a case where the cap 34 is mounted on the distal end portion main body 32, an elevator accommodation space (not shown) is defined by the partition wall 68 and a wall portion 34B of the cap 34. A through-hole 61 is formed in the distal end portion main body 32, and the wire 38 is inserted into the through-hole 61.

On an upper surface 68A of the partition wall 68 that is on a Z (+) side, the illumination window 74 and the observation window 76 are disposed adjacent to each other in a Y direction. Through the illumination window 74, it is possible to irradiate a visual field region in the Z (+) direction with illumination light and through the observation window 76, it is possible to observe the visual field region in the Z (+) direction. Note that, the distal end portion main body 32 is provided with the air/water supply nozzle 58 facing the observation window 76 and the observation window 76 is washed with air and water jetted from the air/water supply nozzle 58.

Next, the cap 34 will be described.

The cap 34 is formed of an elastic material such as a rubber material or a resin material. Examples of the rubber material include fluororubber and silicon rubber, and examples of the resin material include polysulfone and polycarbonate.

The cap 34 includes the wall portion 34B of which a distal end side is sealed and is formed in a substantially tubular shape, and a substantially rectangular opening window 34A is formed in a part of the wall portion 34B. The opening window 34A is open in the Z (+) direction.

A bearing 34C that rotatably supports the elevator 36 is provided in the cap 34. The bearing 34C is configured as a plate-like body that has a height in the Z (+) direction and that extends in the Y (+) direction.

The elevator 36 includes a rotary shaft 36B extending along an X direction, and the rotary shaft 36B is rotatably supported at a through-hole (not shown) of the bearing 34C. Accordingly, the elevator 36 is rotated about the rotary shaft 36B so that the posture thereof is changed between a laid-down position (refer to FIG. 2) and an elevation position.

A distal end portion of the wire 38 is connected to the elevator 36. The wire 38 is connected to a position that is on the distal end side of the elevator 36, that is opposite to a side on which the rotary shaft 36B is formed, and that is adjacent to the treatment tool guide surface 36A.

The cap 34 configured as described above is a type of cap to which the elevator 36 is attached in advance, and the wire 38 is also connected to the elevator 36 in advance. In a case where a treatment performed by means of the endoscope 10 is finished, the cap 34 in the present example is removed from the distal end portion main body 32 and is discarded together with the elevator 36 and the wire 38 as, for example, a disposable. Note that the elevator 36 may be attached to the distal end portion main body 32 instead of being attached to the cap 34.

<First Embodiment>

Hereinafter, the wire fixation mechanism 78 of the first embodiment which is shown in FIG. 1 will be described. The wire fixation mechanism 78 includes the sliding lever 80 and the fixation unit 82 as described above.

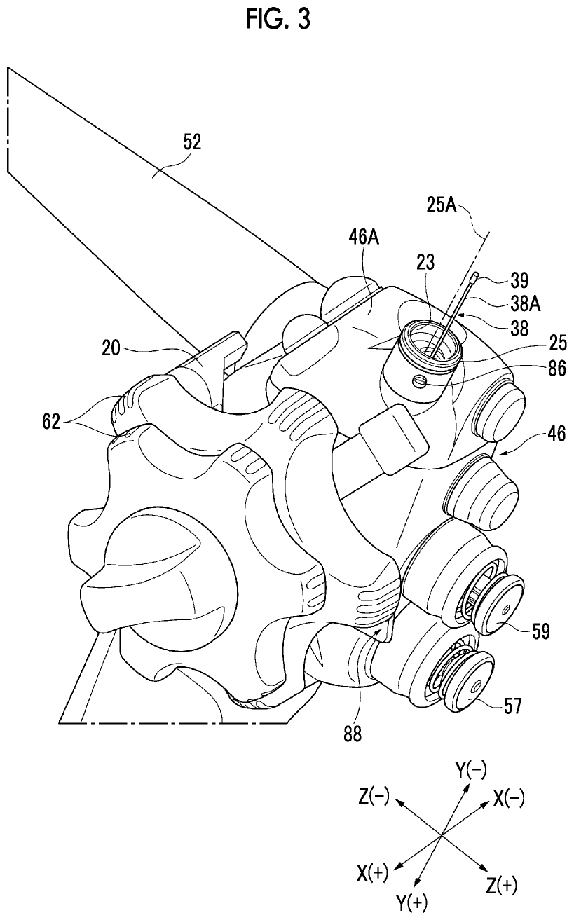

First, a configuration and a procedure for mounting the fixation unit 82 on the operation portion main body 46 will be described with reference to FIGS. 3 to 8. Each of FIGS. 3 to 8 is an enlarged perspective view showing a portion of the operation portion main body 46 that is on a proximal end side.

As shown in FIG. 3, a proximal end surface 46A of the operation portion main body 46 is provided with a cylindrical connection portion 25 in which an outlet port 23 for extraction of the proximal end side of the wire 38 is formed. The connection portion 25 protrudes in the Y (−) direction from the proximal end surface 46A and the proximal end side of the wire 38 protrudes in the Y (−) direction from the outlet port 23 thereof. Note that the wire 38 protrudes from a position eccentric with respect to an axis 25A of the connection portion 25.

The wire 38 includes a long wire main body 38A and a locking target portion 39 that is positioned on a proximal end side of the wire main body 38A and is formed to have an outer shape larger than that of the wire main body 38A. Note that, in FIG. 3, a cylindrical shape is shown as the shape of the locking target portion 39. However, the present invention is not limited thereto and a spherical shape may also be adopted as long as the outer shape of the locking target portion 39 is larger than that of the wire main body 38A. Note that, in the following description, “the wire 38” mainly refers to the wire main body 38A.

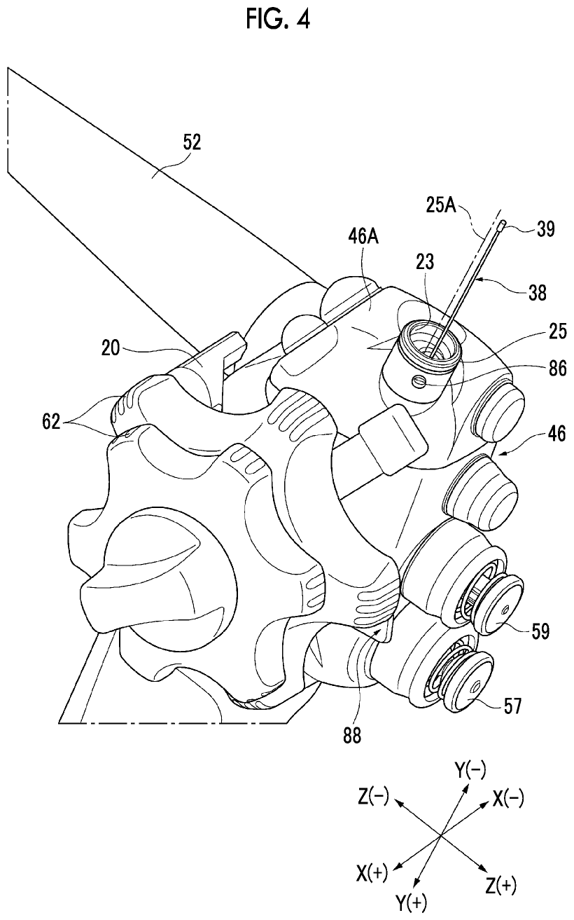

Here, the length of protrusion of the wire 38 protruding from the connection portion 25 will be briefly described. FIG. 4 shows the wire 38 of which the length of protrusion is larger than that of the wire 38 shown in FIG. 3. The wires 38 shown in FIGS. 3 and 4 have the same length as each other. The difference in length of protrusion between the wires 38, which have the same length as each other, is attributable to the state of the soft portion 26 (refer to FIG. 1) or the bendable portion 28.

That is, in a case where the soft portion 26 is in a looped state or the bendable portion 28 is in a bent state, the wire channel 40 (refer to FIG. 2) with the wire 38 inserted thereinto extends and the length of an insertion path of the wire 38 is enlarged. For this reason, the wire 38 becomes relatively short with respect to the insertion path of the wire 38, which results in a small length of protrusion as shown in FIG. 3. On the other hand, in a case where the soft portion 26 or the bendable portion 28 is in a straight state, the wire channel 40 does not extend, which results in a large length of protrusion as shown in FIG. 4. Note that the wire fixation mechanism 78 of the present example has a configuration in which the wire 38 can be fixed without being influenced by the length of protrusion of the wire 38 but this configuration will be described later.

Hereinafter, as an example, a case where the wire fixation mechanism 78 is mounted to the operation portion main body 46 shown in FIG. 4 will be described.

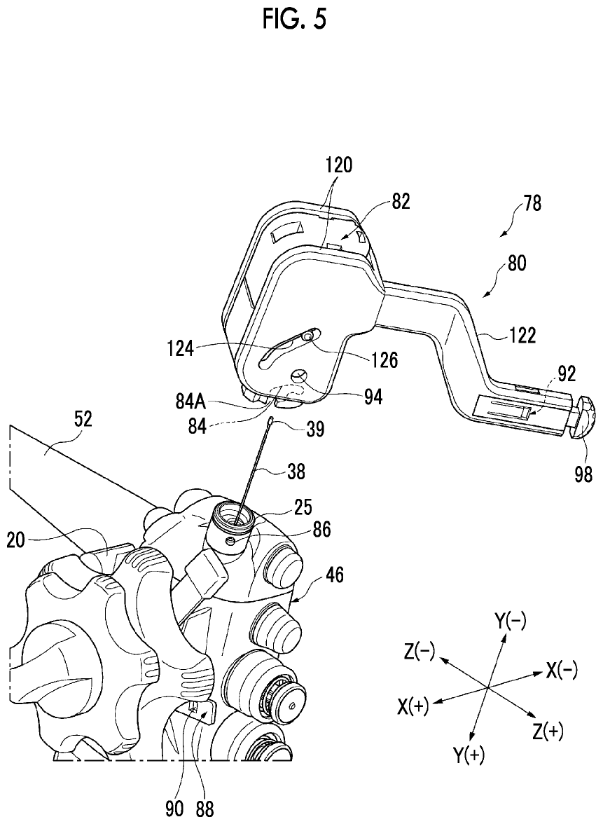

First, as shown in FIG. 5, the fixation unit 82 is caused to face the locking target portion 39 of the wire 38. At this time, in the Y-axis direction parallel to the wire axis direction, an opening end 84A of a cam groove 84 provided at the fixation unit 82 is aligned with a cam pin 86 protruding from an outer peripheral surface of the connection portion 25. The cam groove 84 is formed to be inclined in the Y (−) direction from the opening end 84A.

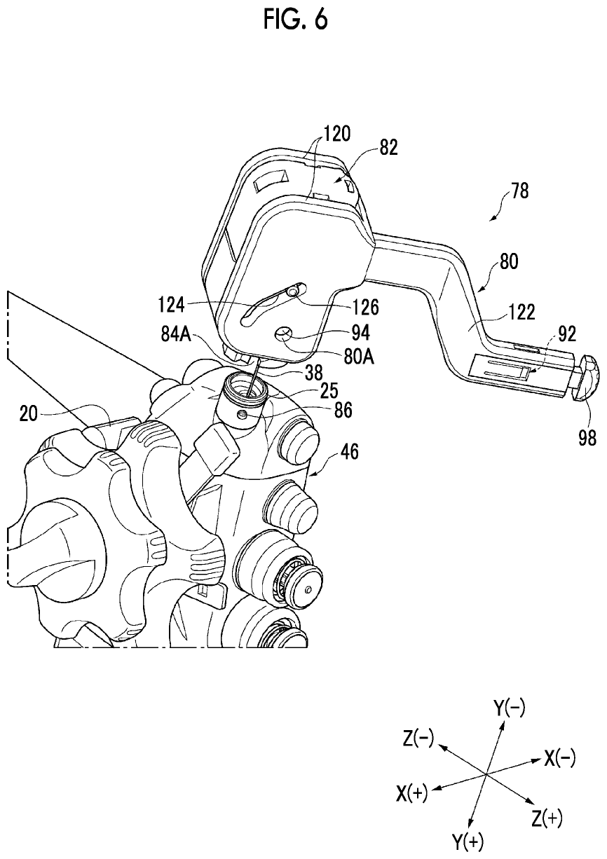

Next, as shown in FIG. 6, while the fixation unit 82 is caused to advance toward the connection portion 25 in the Y (+) direction, the wire 38 is accommodated into the fixation unit 82 with the locking target portion 39 being at the front. Hereinafter, such an operation will be referred to as a “wire accommodation operation”.

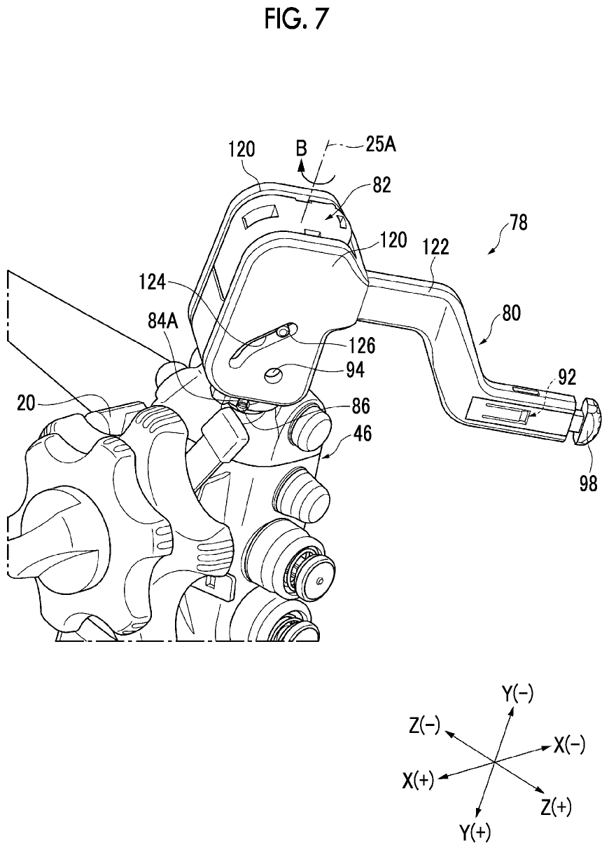

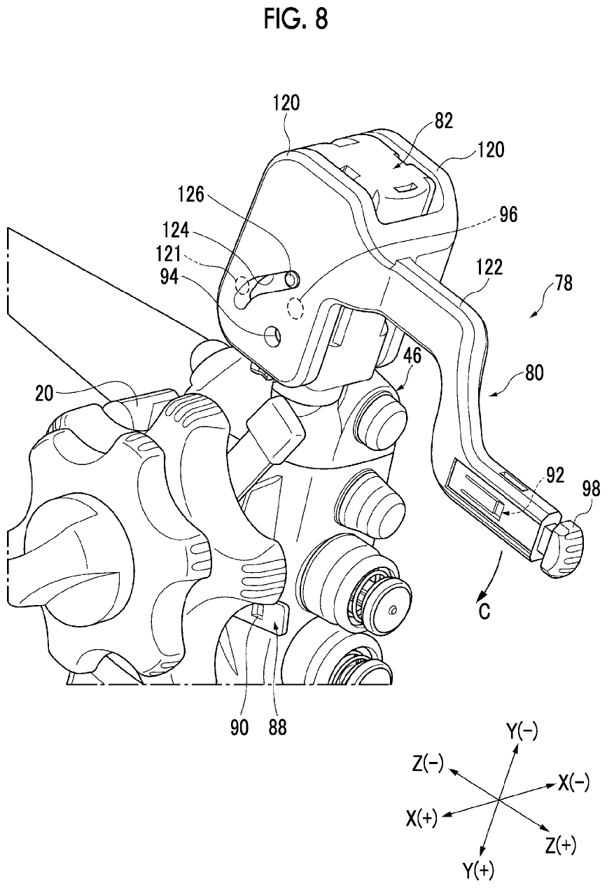

Next, as shown in FIG. 7, in a case where the cam pin 86 is accommodated into the opening end 84A of the cam groove 84 (refer to FIG. 5), the fixation unit 82 in a state as shown in FIG. 7 is caused to rotate in a clockwise direction represented by an arrow B with the axis 25A (refer to FIG. 4) as a rotation axis, the axis 25A being eccentric from the wire 38. In this case, it is preferable that the fixation unit 82 is rotated by means of the sliding lever 80. As a result, the fixation unit 82 is pushed in the Y (+) direction by a guide action of the cam groove 84 and the cam pin 86. Then, the fixation unit 82 is mounted on the operation portion main body 46 via the connection portion 25 in a posture as shown in FIG. 8 in which the cam pin 86 has reached a terminal end of the cam groove 84. Hereinafter, such an operation will be referred to as a “rotational mounting operation”. Therefore, the fixation unit 82 is mounted on the operation portion main body 46 through the above-described “wire accommodation operation” and “rotational mounting operation”. Note that the “wire accommodation operation” and the “rotational mounting operation” are performed by means of one action.

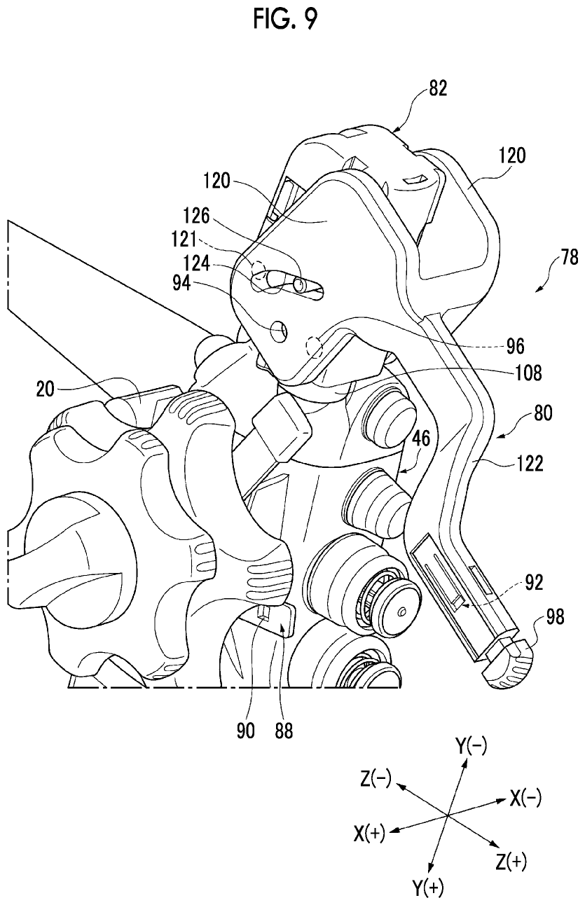

Next, a configuration for connection of the sliding lever 80 to the elevation operation lever 20 side and a procedure thereof will be described with reference to FIG. 9. FIG. 9 is an enlarged perspective view showing a portion of the operation portion main body 46 that is on the proximal end side.

As shown in FIG. 9, the operation portion main body 46 includes a link member 88 connected to the elevation operation lever 20. The link member 88 is provided to be rotatable around a rotation axis of the elevation operation lever 20 and is rotated, as the elevation operation lever 20 is rotated, in the same direction as the elevation operation lever 20. An opening portion 90 is formed in the link member 88, and the sliding lever 80 is attachably and detachably connected to the elevation operation lever 20 via the link member 88 in a case where a claw portion 92 provided at the sliding lever 80 is engaged with the opening portion 90. The claw portion 92 is provided at a distal end (a free end) of a cantilevered elastic piece provided at the sliding lever 80 and functions as a lever connecting portion according to the embodiment of the present invention.

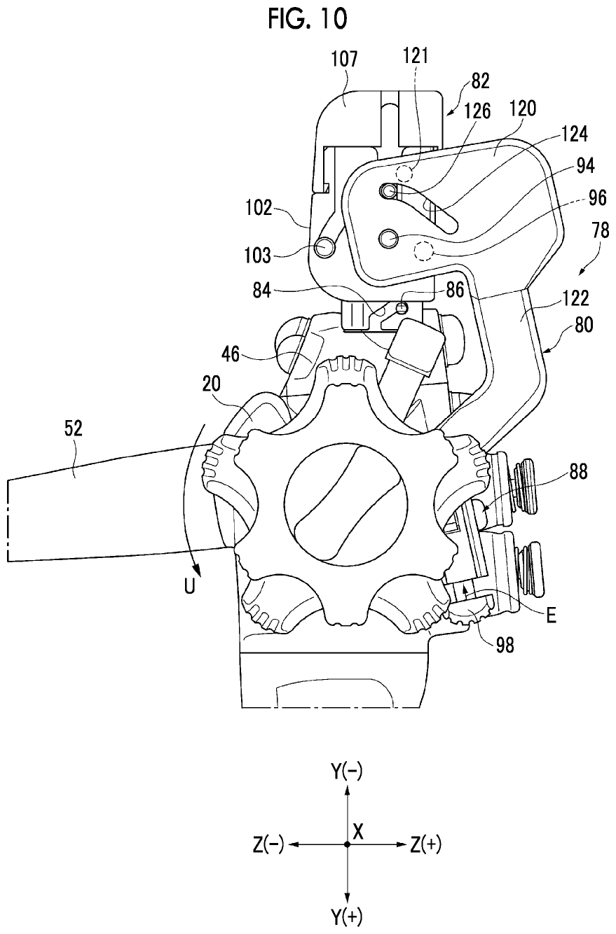

Meanwhile, the sliding lever 80 is rotatably connected to the fixation unit 82 via first shafts 94 and second shafts 96 represented by broken lines, the first shafts 94 and the second shafts 96 being selectively switched. Although the details will be described later, in a case where the sliding lever 80 shown in FIG. 8 is pushed down toward the link member 88 in a direction represented by an arrow C, the sliding lever 80 rotates with the first shafts 94 as a rotation axis thereof first and approaches the link member 88 as shown in FIG. 9. In a case where a pushing operation as described above is continued thereafter, the sliding lever 80 rotates with the second shafts 96 as a rotation axis thereof and thus the claw portion 92 is engaged with the opening portion 90 (refer to FIG. 9) as shown in FIG. 10. Hereinafter, such an operation will be referred to as a “lever connection operation”. Therefore, the sliding lever 80 is connected to the elevation operation lever 20 side through the “wire accommodation operation”, the “rotational mounting operation”, and the “lever connection operation” which have been described above. In such a manner, the wire fixation mechanism 78 is mounted to the operation portion main body 46. FIG. 10 is a front view of the operation portion main body 46 as seen in a case where the operation portion main body 46 is seen from an X (+) direction side.

Note that FIG. 10 shows a state in which the elevation operation lever 20 is positioned at a laying down operation position. That is, the wire fixation mechanism 78 of the present example is connected to the elevation operation lever 20 positioned at the laying down operation position via the link member 88. In addition, although the details will be described later, the locking target portion 39 (refer to FIG. 4) of the wire 38 is fixed to the fixation unit 82 through the “wire accommodation operation”, the “rotational mounting operation”, and the “lever connection operation” which have been described above. Note that, in the embodiment, a case where the lever connection operation is performed at the laying down operation position has been described as one of preferred embodiments. However, the present invention is not limited thereto, and the lever connection operation may be performed at a position other than the laying down operation position. For example, the lever connection operation may be performed at an elevation operation position or at a position between the elevation operation position and the laying down operation position.

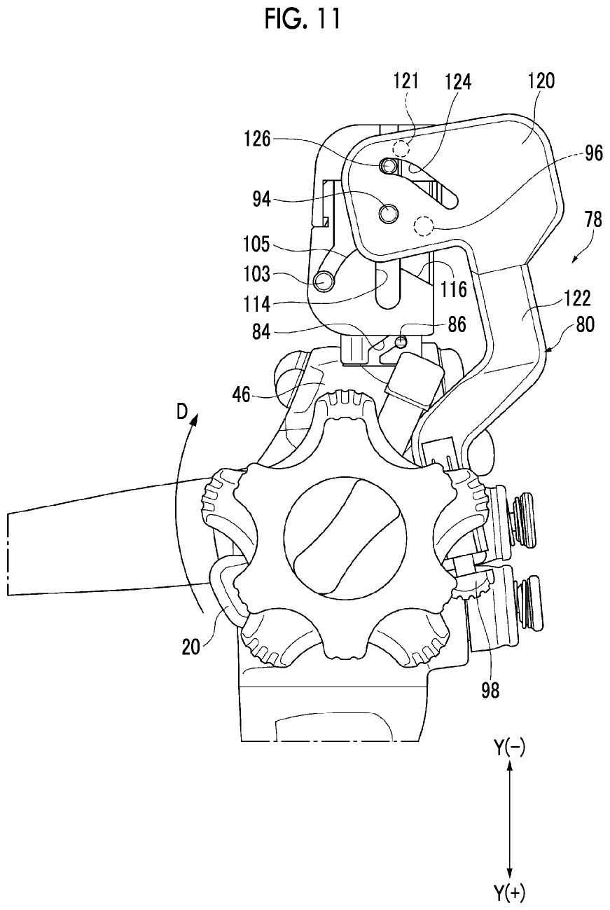

In FIG. 10, in a case where the posture of the elevator 36 (refer to FIG. 2) is to be changed by rotating the elevation operation lever 20, the elevation operation lever 20 as shown in FIG. 10 that is positioned at the laying down operation position is rotated toward the elevation operation position shown in FIG. 11 in a counterclockwise direction represented by an arrow U (refer to FIG. 10). Then, the link member 88 rotates in the counterclockwise direction, the sliding lever 80 connected to the link member 88 moves in the Y (− direction, and the fixation unit 82 connected to the sliding lever 80 moves in the Y (−) direction. Since the locking target portion 39 of the wire 38 (refer to FIG. 2) is fixed to the fixation unit 82, the wire 38 is pulled in the Y (−) direction as the elevation operation lever 20 is rotated as described above. Accordingly, the posture of the elevator 36 connected to the distal end portion of the wire 38 is changed from the laid-down position in FIG. 2 to the elevation position.

On the contrary, in a case where the elevator 36 is to be laid down, the elevation operation lever 20 in FIG. 11 positioned at the elevation operation position is rotated toward the laying down operation position shown in FIG. 10 in a clockwise direction represented by an arrow D (refer to FIG. 11). Then, the link member 88 (refer to FIG. 9) rotates in the clockwise direction, the sliding lever 80 connected to the link member 88 moves in the Y (+) direction, and the fixation unit 82 connected to the sliding lever 80 moves in the Y (+) direction. Accordingly, the wire 38 is pushed in the Y (+) direction, and the posture of the elevator 36 is changed from the elevation position to the laid-down position in FIG. 2.

Note that, in a case where the sliding lever 80 and the link member 88 are to be disconnected from each other, an unlocking member 98 that protrudes at a distal end of the sliding lever 80 is pushed toward the link member 88 in a direction along an arrow E, as shown in FIG. 10. Accordingly, the claw portion 92 is pushed by the unlocking member 98 and is withdrawn from the opening portion 90, so that the above-described connection state can be eliminated.

In addition, in a case where the wire fixation mechanism 78 is to be removed from the operation portion main body 46, the “lever connection operation”, the “rotational mounting operation”, and the “wire accommodation operation” may be performed reversely.

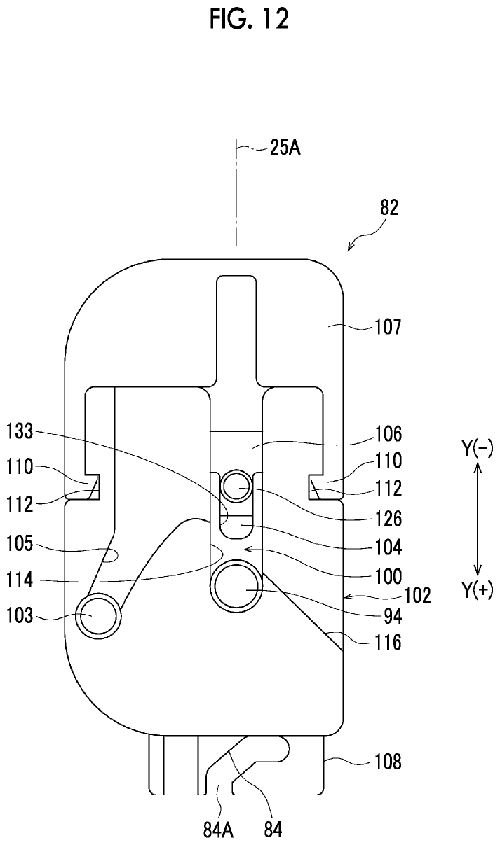

Next, the fixation unit 82 will be described. FIG. 12 is a front view of the fixation unit 82.

As shown in FIG. 12, the fixation unit 82 includes a wire catch 100 that attachably and detachably locks and fixes a proximal end side of the wire 38 and a catch guide 102 that guides the wire catch 100 in the wire axis direction (the Y-axis direction) of the wire 38. In addition, the wire catch 100 includes a catch main body 104 and a fixing member 106. The fixing member 106 functions as a fixation portion according to the embodiment of the present invention. Note that although details will be described later, among the constituent members of the fixation unit 82, a member that moves in the Y-axis direction as the sliding lever 80 is operated (the lever connection operation and a driving operation of the elevator 36) is the wire catch 100 and the catch guide 102 does not move.

The catch guide 102 includes a cylindrical connection portion 108 that includes the cam groove 84 and that is provided at an end portion on a Y (+) direction side, and the connection portion 108 is connected to the connection portion 25 of the operation portion main body 46 (refer to FIG. 6). In addition, regarding the catch guide 102, a cap 107 is attached to an end portion on a Y (−) direction side. Claw portions 110 and 110 are formed on both walls of the cap 107 and the cap 107 is attachably and detachably attached to the catch guide 102 with the claw portions 110 and 110 engaged with grooves 112 and 112 on both walls of the catch guide 102.

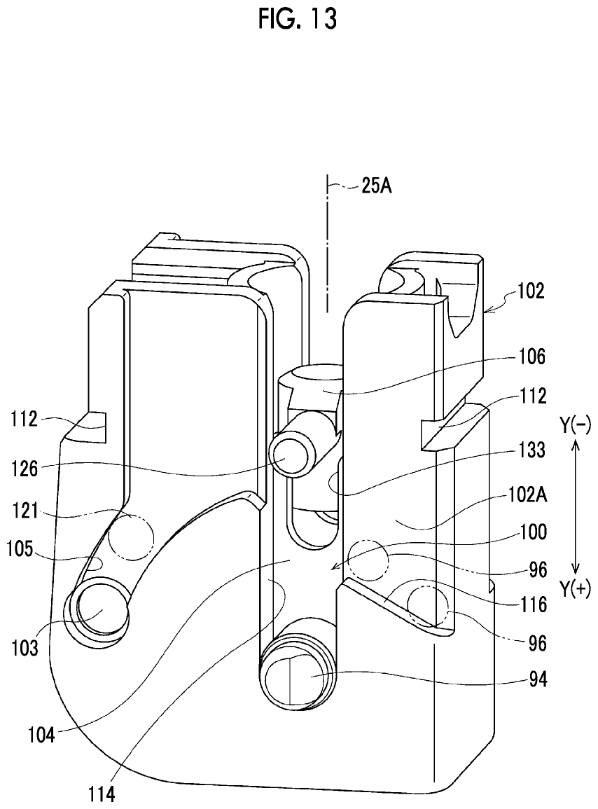

FIG. 13 is a main part perspective view in the case of removal of the cap 107 from the catch guide 102 shown in FIG. 12.

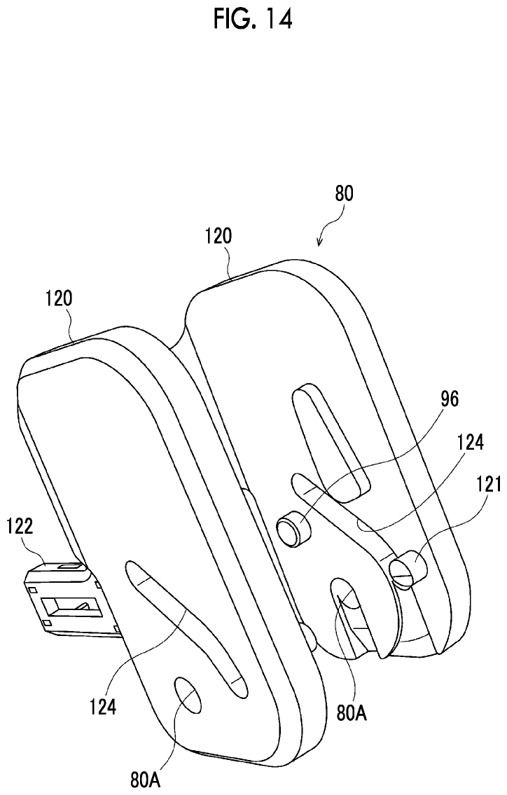

As shown in FIG. 13, a catch guide groove 114 extending in the wire axis direction is formed in a central portion of the catch guide 102 and the first shafts 94 are slidably engaged and guided along the catch guide groove 114. As described above, each of the first shafts 94 is a shaft that constitutes one of rotary shafts of the sliding lever 80, and is fixed to the catch main body 104. Lever bearing holes 80A (refer to FIG. 14) of the sliding lever 80 is rotatably engaged with the first shafts 94.

Here, the configuration of the sliding lever 80 will be described briefly. FIG. 14 is a perspective view showing a main part of the sliding lever 80.

As shown in FIG. 14, the sliding lever 80 includes a pair of plate-shaped portions 120 and 120 that holds the fixation unit 82 (refer to FIG. 5) with the fixation unit 82 interposed between the plate-shaped portions, and a lever main body 122 that is integrated with the plate-shaped portions 120 and 120.

The second shaft 96, which is one of the rotary shafts of the sliding lever 80, is provided on each of inner surfaces of the plate-shaped portions 120 and 120 that face each other, and the second shafts 96 protrude toward surfaces 102A of the catch guide 102 shown in FIG. 13. In addition, substantially L-shaped cam grooves 124 are formed on the plate-shaped portions 120 and 120 while being positioned to face each other and a pin 126 provided in the fixing member 106 (refer to FIG. 13) is engaged with the cam grooves 124. The cam grooves 124 will be described later.

Further, bosses 121 are provided on the inner surfaces of the plate-shaped portions 120 and 120 that face each other, and the bosses 121 protrude toward the surfaces 102A of the catch guide 102 shown in FIG. 13. In addition, boss holes 103 with which the bosses 121 are elastically engaged are formed in the surfaces 102A. Therefore, the posture of the sliding lever 80 as shown in FIGS. 5 to 8 is maintained with the bosses 121 engaged with the boss holes 103. In addition, in a case where the bosses 121 are disengaged from the boss holes 103, the sliding lever 80 is allowed to rotate.

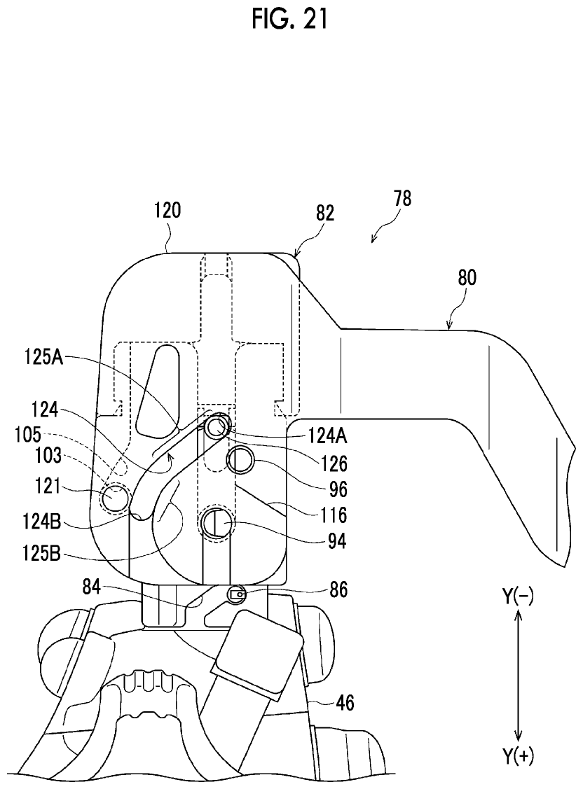

In addition, at the surfaces 102A of the catch guide 102, first restriction surfaces 105 that the bosses 121 represented by two-dot chain lines in FIG. 13 can abut are formed. The first restriction surfaces 105 abut the bosses 121 to restrict movement of the sliding lever 80 in a case where the pin 126 is present in first cam groove portions 125A (which will be described later) (refer to FIG. 21) of the cam grooves 124. Since the first restriction surfaces 105 are provided, the sliding lever 80 can rotate around the first shafts 94. In addition, the first restriction surfaces 105 are composed of arc-shaped surfaces centered on the first shafts 94. Accordingly, the sliding lever 80 can smoothly rotate around the first shafts 94.

In addition, second restriction surfaces 116 are formed at the surfaces 102A of the catch guide 102. The second restriction surfaces 116 are surfaces that can abut the second shafts 96 in a case where the sliding lever 80 restricted by the first restriction surfaces 105 is released. That is, the first restriction surfaces 105 are formed only up to positions corresponding to positions where the second shafts 96 abut the second restriction surfaces 116 and the sliding lever 80 restricted by the first restriction surfaces 105 is released at the positions where the second shafts 96 abut the second restriction surfaces 116. Note that the sliding lever 80 restricted by the first restriction surfaces 105 may be released at the same time as when the second shafts 96 abut the second restriction surfaces 116 and may be released before or after the second shafts 96 abut the second restriction surfaces 116. In addition, the second restriction surfaces 116 abut the second shafts 96 to restrict movement of the sliding lever 80 in a case where the pin 126 is present in second cam groove portions 125B (which will be described later) (refer to FIG. 21) of the cam grooves 124. Since the second restriction surfaces 116 are provided, the sliding lever 80 can rotate around the second shafts 96 with the second shafts 96 being moved along the second restriction surfaces 116.

The second restriction surfaces 116 are formed to be inclined in the Y (+) direction from the catch guide groove 114 toward the outside of the catch guide 102. In a case where the second shafts 96 abut the second restriction surfaces 116 and move, the rotation axis of the sliding lever 80 is switched from the first shafts 94 to the second shafts 96 during the “lever connection operation” of the sliding lever 80.

Here, a rotation axis switching operation will be described. In the first half of the “lever connection operation”, the sliding lever 80 rotates with the first shafts 94 as the rotation axis thereof. At this time, as represented by two-dot chain lines in FIG. 13, the second shafts 96 move toward the second restriction surfaces 116 from positions on a left side with respect to the second restriction surfaces 116. Then, at the end of the first half of the “lever connection operation”, the first shafts 94 restricted by the first restriction surfaces 105 are released and the second shafts 96 abut the second restriction surfaces 116 so that the second restriction surfaces 116 restrict the movement of the sliding lever 80. In addition, in the second half of the “lever connection operation”, rotation around the second shafts 96 is possible and thus the sliding lever 80 rotates with the second shafts 96 as the rotation axis thereof. The above-described switching operation is as described above.

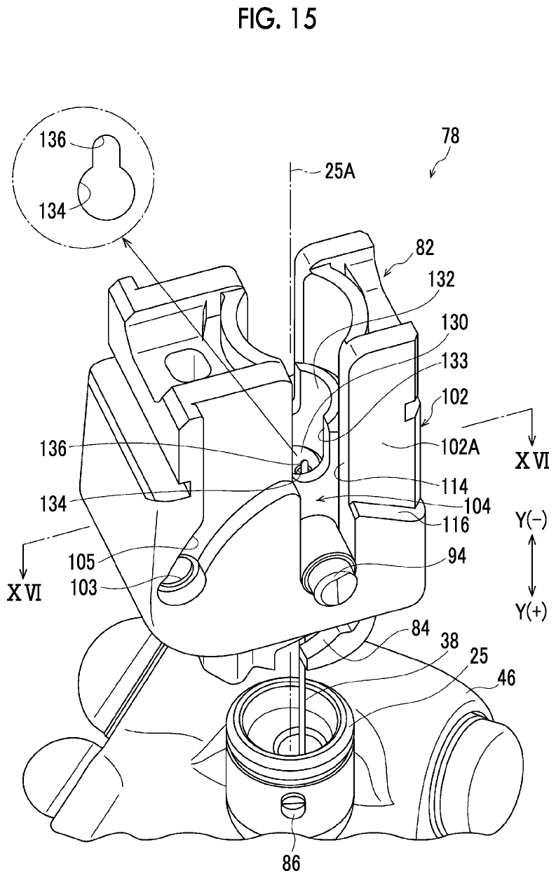

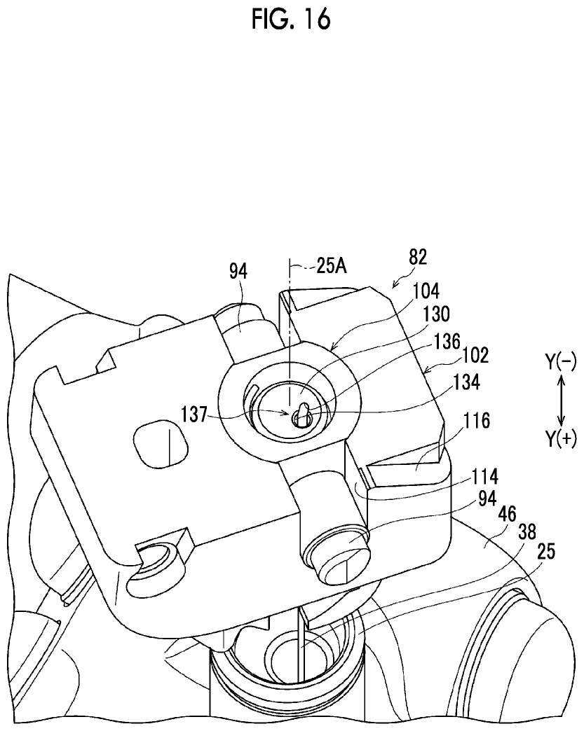

Next, the catch main body 104 of the wire catch 100 will be described. FIG. 15 is a perspective view showing a state immediately before the wire fixation mechanism 78 is mounted to the operation portion main body 46. FIG. 16 is a cross-sectional view of the fixation unit 82 which is taken along line XVI-XVI of FIG. 15.

As shown in FIGS. 15 and 16, the catch main body 104 includes a columnar portion 130, a pair of the first shafts 94 and 94 that protrudes from the columnar portion 130 in a direction orthogonal to an axis (that coincides with the axis 25A which is the rotation axis of the fixation unit 82) of the columnar portion 130, and a guide portion 132 that protrudes from the columnar portion 130 in the Y (−) direction. A catch main body groove 133 extending in the wire axis direction is formed in the guide portion 132. The catch main body groove 133 is provided at a position overlapping with the catch guide groove 114, and the pin 126 (refer to FIG. 13) is inserted into the catch main body groove 133. The pin 126 is a cam pin that is engaged with the cam grooves 124 of the sliding lever 80, and is guided by the cam grooves 124 to be movable forward and backward along the catch main body groove 133.

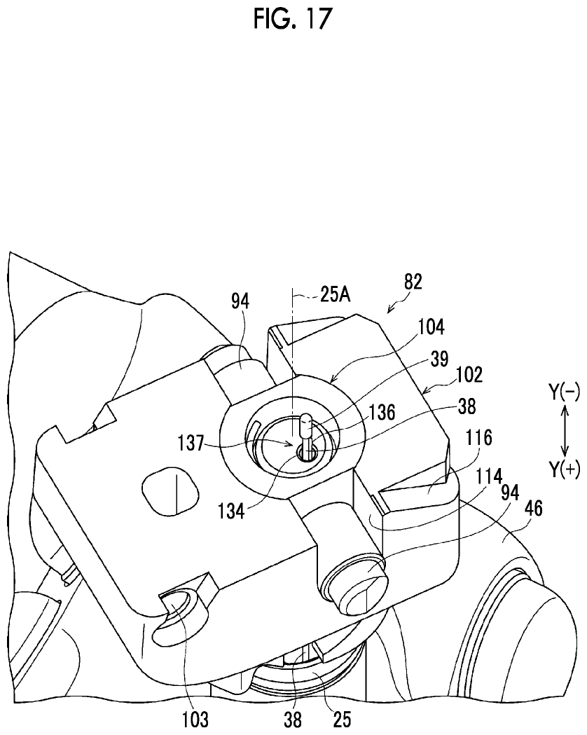

As shown in FIG. 16, the columnar portion 130 includes a locking hole 137 into which the locking target portion 39 (refer to FIG. 17) is insertable and at which the locking target portion 39 is lockable. The locking hole 137 is formed as a through-hole that penetrates the columnar portion 130 in the Y-axis direction.

The locking hole 137 includes a first hole 134 of which the size is enough for the locking target portion 39 to be inserted thereinto and a second hole 136 of which the outer shape is larger than that of the wire main body 38A and is smaller than that of the locking target portion 39 and the locking hole 137 has an opening shape in which the first hole 134 and the second hole 136 are continuously connected to each other.

In addition, the locking hole 137 is provided at a position eccentric from the axis 25A which is the rotation axis of the fixation unit 82 and the first hole 134 and the second hole 136 are formed to be continuously connected to each other along the trajectory of rotation around the axis 25A. Note that the amount of eccentricity of the locking hole 137 with respect to the axis 25A is set to be substantially equal to the amount of eccentricity of the wire 38 with respect to the axis 25A shown in FIG. 4.

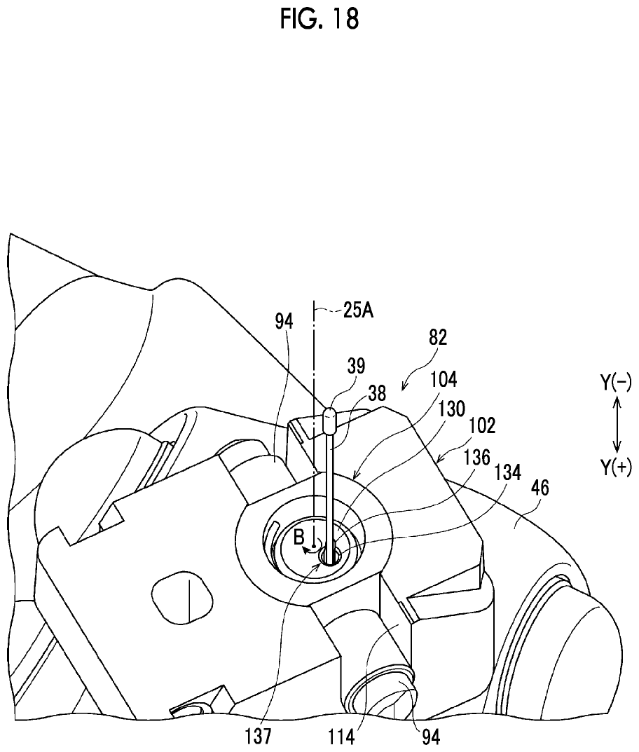

According to the catch main body 104 configured as described above, the locking target portion 39 is accommodated into the first hole 134 in the case of the “wire accommodation operation” (refer to FIG. 6). Then, the locking target portion 39 passes through the first hole 134 and protrudes to the outside from the first hole 134 as shown in FIG. 17. Then, at the end of the “wire accommodation operation” (refer to FIG. 7), the wire 38 protrudes from the first hole 134 in the Y (−) direction as shown in the cross-sectional view in FIG. 18.

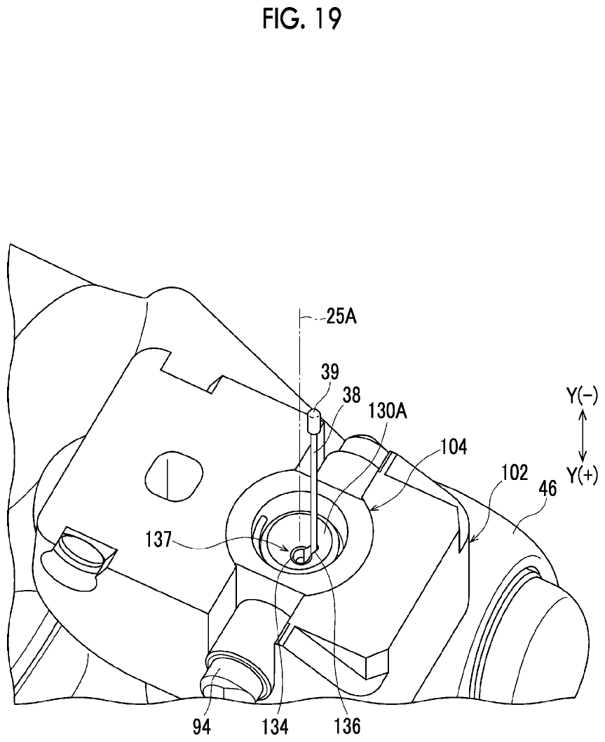

Thereafter, in the case of the “rotational mounting operation” (refer to FIG. 7), the catch main body 104 rotates around the axis 25A in a direction along the arrow B together with the catch guide 102, so that the first hole 134 is withdrawn from the wire 38. Then, at the end of the “rotational mounting operation”, the wire 38 is accommodated into the second hole 136 as shown in a cross-sectional view in FIG. 19. Accordingly, the locking target portion 39 can be locked at the second hole 136.

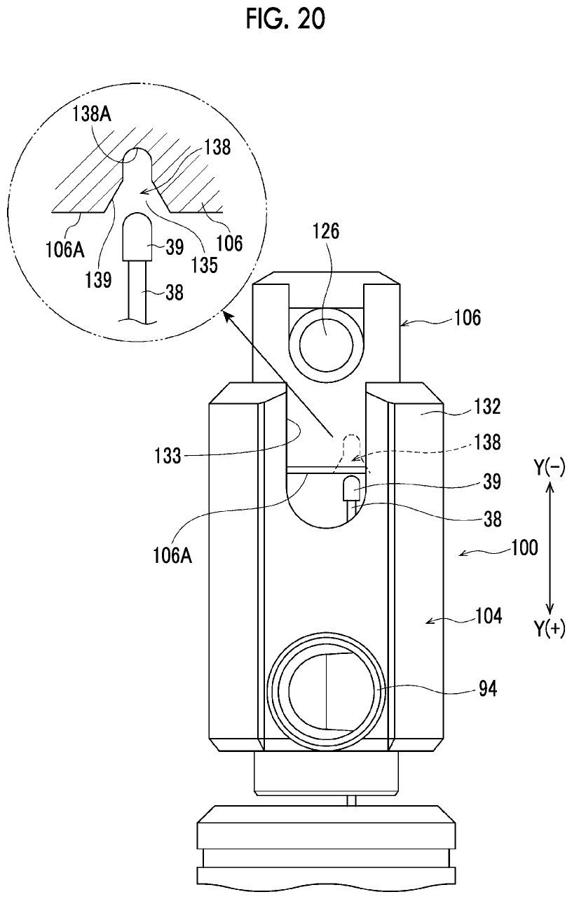

FIG. 20 is an explanatory view showing an example of the positional relationship between the catch main body 104 and the fixing member 106 at the end of the “rotational mounting operation” shown in FIG. 19.

As shown in FIG. 20, the fixing member 106 is disposed on the Y (−) direction side with respect to the catch main body 104. A fixation hole 138, in which an opening portion 135 into which the locking target portion 39 is accommodable is formed, is formed in an end surface 106A of the fixing member 106 that is on the Y (+) direction side.

The fixation hole 138 is formed at a position facing the second hole 136 shown in FIG. 19 in the Y-axis direction, and a bottom portion 138A that is engaged with the locking target portion 39 is provided in the fixation hole 138. In addition, the fixation hole 138 includes a conical guide surface 139 that becomes narrower from the opening portion 135 toward the bottom portion 138A. Although the guide surface 139 is not essential, it is preferable that the fixation hole 138 includes the guide surface 139 in a viewpoint of smoothly guiding the locking target portion 39 to the bottom portion 138A. Note that, as shown in FIG. 20, at the end of the “rotational mounting operation” (that is, before the start of the “lever connection operation”), the locking target portion 39 is not engaged with the bottom portion 138A and is positioned at a position on the Y (+) direction side while separated from the bottom portion 138A.

FIG. 21 is a front view of the wire fixation mechanism 78 at the end of the “rotational mounting operation” shown in FIG. 19, and is a see-through view showing the plate-shaped portions 120 of the sliding lever 80 through a view.

As shown in FIG. 21, at the end of the “rotational mounting operation”, the bosses 121 are fitted to the boss holes 103, the second shafts 96 are positioned on an upper left side in FIG. 21 with respect to the second restriction surfaces 116, and the pin 126 is positioned at right ends 124A of the cam grooves 124.

Here, the cam grooves 124 will be described. Each cam groove 124 has a shape in which the linear first cam groove portion 125A and a curved second cam groove portion 125B are continuously connected to each other. The first cam groove portion 125A has a function of changing a relative distance between the catch main body 104 and the fixing member 106 by moving the fixing member 106 in the Y-axis direction in cooperation with the pin 126. In addition, the second cam groove portion 125B has a function of maintaining the relative distance between the catch main body 104 and the fixing member 106 by moving the fixing member 106 integrally with the catch main body 104 in the Y-axis direction.

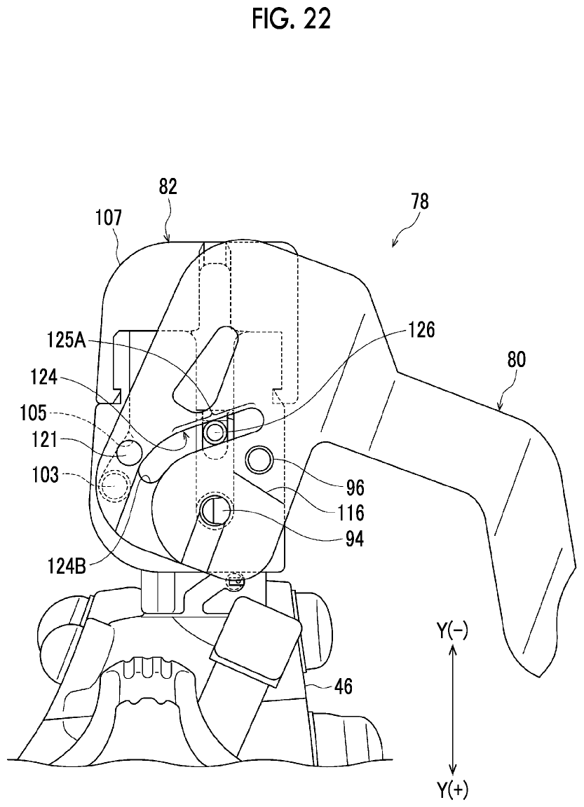

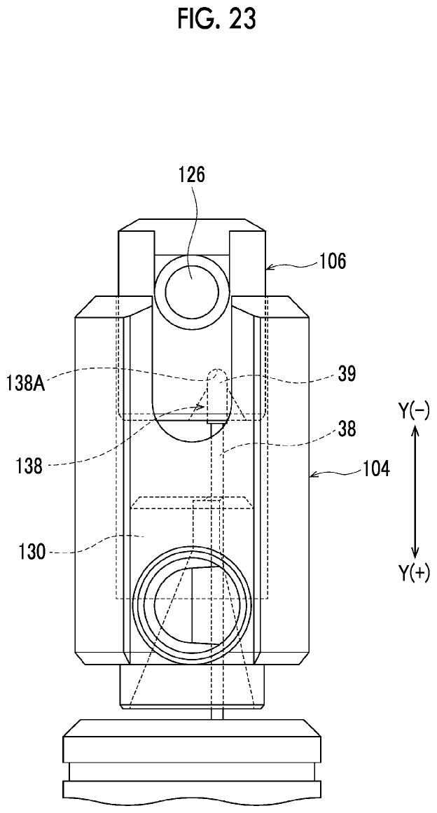

Specifically, in a case where the “lever connection operation” is started in a state as shown in FIG. 21, the bosses 121 are separated from the boss holes 103 and guided to the first restriction surfaces 105 as shown in FIG. 22, and the sliding lever 80 rotates in a clockwise direction in FIG. 22 with the first shafts 94 as a rotation axis. Then, because of the rotation, the pin 126 moves along the first cam groove portions 125A. Because of this movement, the fixing member 106 moves in the Y (+) direction and approaches the catch main body 104. Then, as shown in FIG. 23, the bottom portion 138A of the fixation hole 138 of the fixing member 106 is engaged with the locking target portion 39 during the “lever connection operation”.

In addition, in a case where the “lever connection operation” is continued from a position shown in FIGS. 22 and 23, the pin 126 moves along the first cam groove portions 125A and thus the fixing member 106 further moves in the Y (+) direction. Accordingly, the wire 38 is pushed in the Y (+) direction by the fixing member 106.

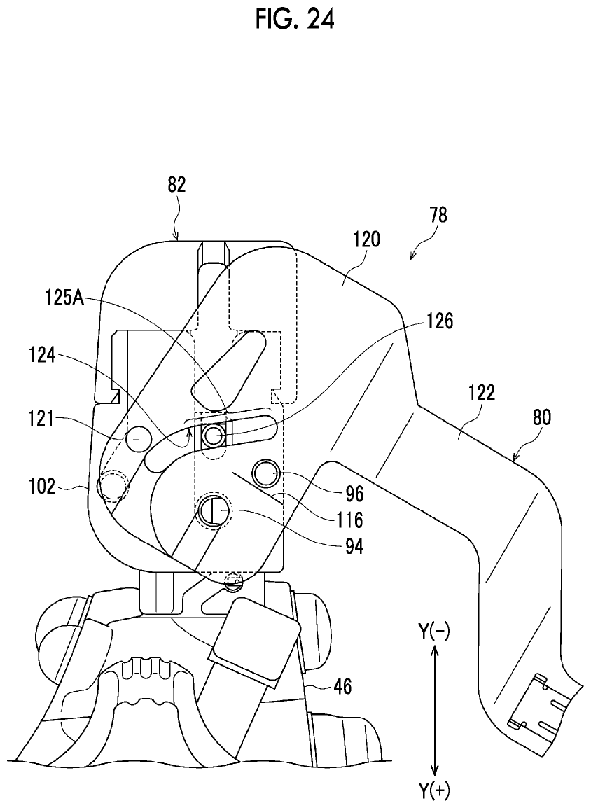

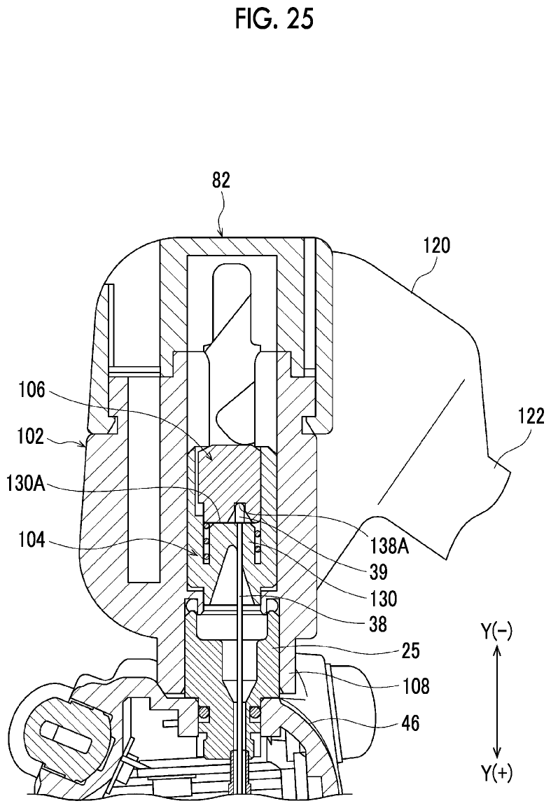

In addition, as shown in FIG. 24, in a case where the second shafts 96 abut the second restriction surfaces 116 (that is, in a case where the first half of the “lever connection operation” is finished), the fixing member 106 abuts the columnar portion 130 of the catch main body 104 as shown in a cross-sectional view in FIG. 25. In this case, the bosses 121 restricted by the first restriction surfaces 105 are released. Accordingly, the locking target portion 39 is locked at the second hole 136 of the catch main body 104 and the locking target portion 39 is interposed between the fixation hole 138 and an end surface 130A of the columnar portion 130 that is on the Y (−) direction side. Because of this operation, a locked state between the locking target portion 39 and the second hole 136 is fixed by the fixing member 106 and the locking target portion 39 is reliably fixed to the fixation unit 82. Note that, the position of the fixing member 106 shown in FIG. 25 is a fixing position, and the position of the fixing member 106 shown in FIG. 20 is an unfixing position. The fixing member 106 can be moved between the fixing position and the unfixing position by means of rotation of the sliding lever 80.

In addition, at the above-described fixing position, the locking target portion 39 is engaged with the bottom portion 138A of the fixation hole 138 and thus movement of the wire 38 in a direction orthogonal to the wire axis direction is restricted by the bottom portion 138A. Accordingly, at the above-described fixing position, movement of the wire 38 from the second hole 136 to the first hole 134 is inhibited and thus the above-described locked state is maintained. Here, an inner wall surface of the bottom portion 138A functions as a restriction surface according to the embodiment of the present invention. The inner wall surface of the bottom portion 138A is at least a portion of an inner wall surface of the fixation hole 138.

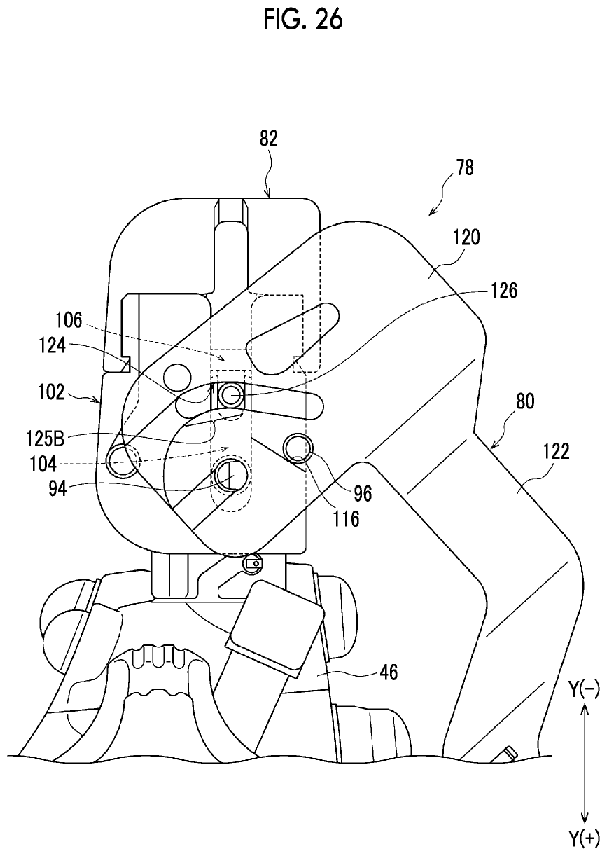

Meanwhile, in a case where the second half of the “lever connection operation” is started from a position in FIG. 24, since the bosses 121 restricted by the first restriction surfaces 105 have been released, the sliding lever 80 rotates around the second shafts 96 in the clockwise direction with the second shafts 96 moving along the second restriction surfaces 116, as shown in FIG. 26. Because of this operation, the catch main body 104 moves in the Y (−) direction via the first shafts 94 and the pin 126 moves along the second cam groove portions 125B, so that the fixing member 106 moves integrally with the catch main body 104 in the Y (−) direction. Because of this operation, the wire 38 pushed in the Y (+) direction is pulled up in the Y (−) direction.

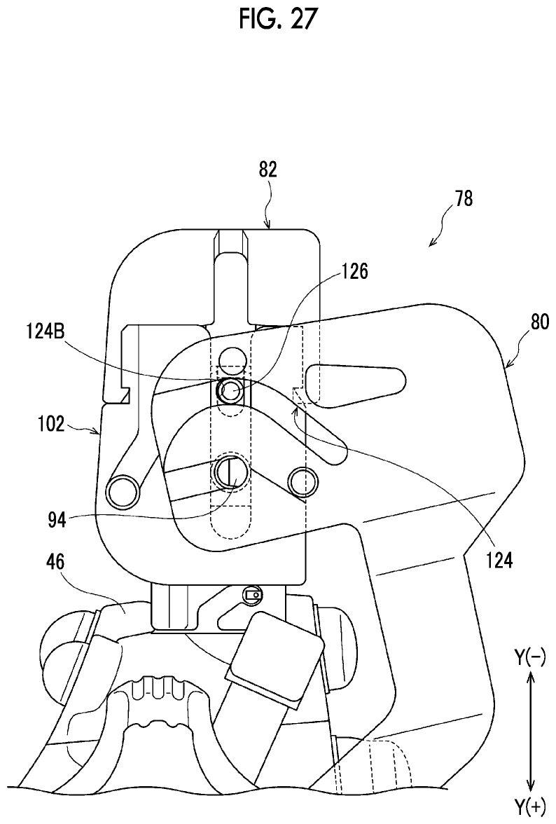

Then, the “lever connection operation” is finished at the laying down operation position in FIG. 27 at which the sliding lever 80 is connected to the link member 88 (refer to FIG. 10), and the above-described movement of the catch main body 104 and the fixing member 106 is stopped. Because of this operation, the proximal end of the wire 38 is pulled up to the laying down operation position of the elevation operation lever 20. At this time, the pin 126 is positioned at left ends 124B of the cam grooves 124. The outline of the operations of the catch main body 104 and the fixing member 106 is as described above. Note that the catch main body 104 and the fixing member 106 reciprocate along the Y-axis direction in a state of abutting each other as the elevation operation lever 20 is rotated (the elevator 36 is driven) thereafter (refer to FIG. 10 and FIG. 11). Accordingly, the wire 38 is pushed and pulled so that the elevator 36 is elevated and laid down.

As described above, according to the endoscope 10 of the present embodiment, since the wire fixation mechanism 78 that fixes the proximal end side of the wire 38 is provided and a configuration in which the locking target portion 39 provided on the proximal end side of the wire 38 is fixed by the fixing member 106 in a state of being locked at the locking hole 137 provided at the wire catch 100 is adopted for the wire fixation mechanism 78, the wire 38 can be reliably fixed to the wire fixation mechanism 78.

Hereinafter, the operation range of the wire fixation mechanism 78 according to the first embodiment will be described.

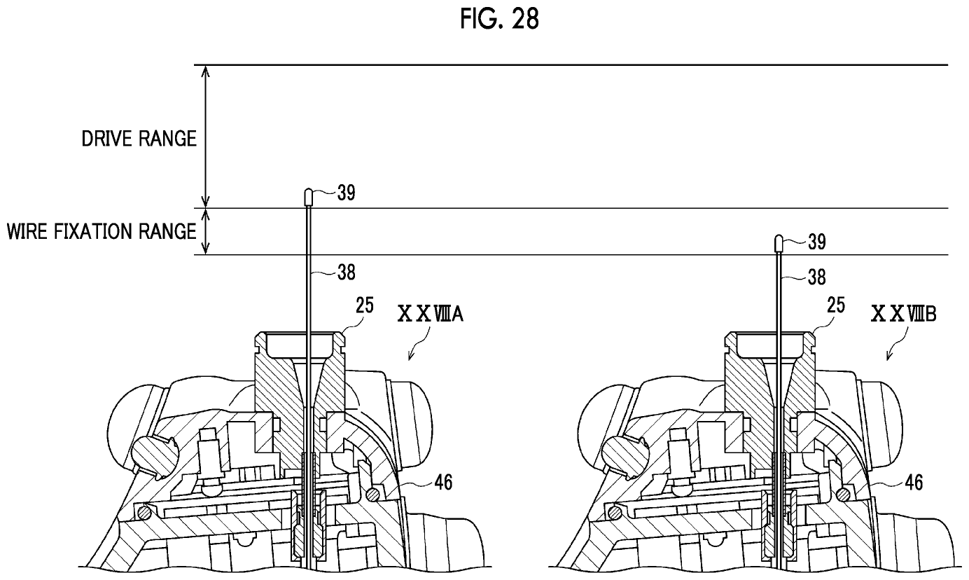

The operation range of the wire fixation mechanism 78 includes a “wire fixation range” in which the wire catch 100 operates because of the “lever connection operation” of the sliding lever 80 and a “drive range” in which the wire catch 100 operates because of rotation of the elevation operation lever 20. FIG. 28 is an explanatory view showing the above-described “wire fixation range” and “drive range”.

As described above, even in the case of the wires 38 having the same length, the length of protrusion of the wires 38 protruding from the connection portion 25 differs depending on the state of the soft portion 26 or the bendable portion 28 (refer to FIG. 1). According to FIG. 28, regardless of whether the length of protrusion of the wire 38 is large (see XXVIIIA of FIG. 28) or the length of protrusion of the wire 38 is small (see XXVIIIB of FIG. 28), with the wire catch 100 operating in the “wire fixation range”, locking the locking target portion 39 with respect to the locking hole 137 and fixing the locked state between the locking target portion 39 and the locking hole 137 are performed by the wire catch 100, so that fixation to the wire fixation mechanism 78 is reliably performed.

Therefore, according to the wire fixation mechanism 78 of the first embodiment, the wire 38 can be reliably fixed regardless of the length of protrusion of the wire 38.

In addition, regarding the wire fixation mechanism 78 of the first embodiment, with the wire catch 100 operating in the “wire fixation range”, the proximal end of the wire 38 can be pulled up to the laying down operation position of the elevation operation lever 20 regardless of the length of protrusion of the wire 38.

Accordingly, according to the wire fixation mechanism 78 of the first embodiment, the positional relationship between the position of the elevator 36 and the position of the elevation operation lever 20 can be made constant regardless of the length of protrusion of the wire 38.

<Second Embodiment>

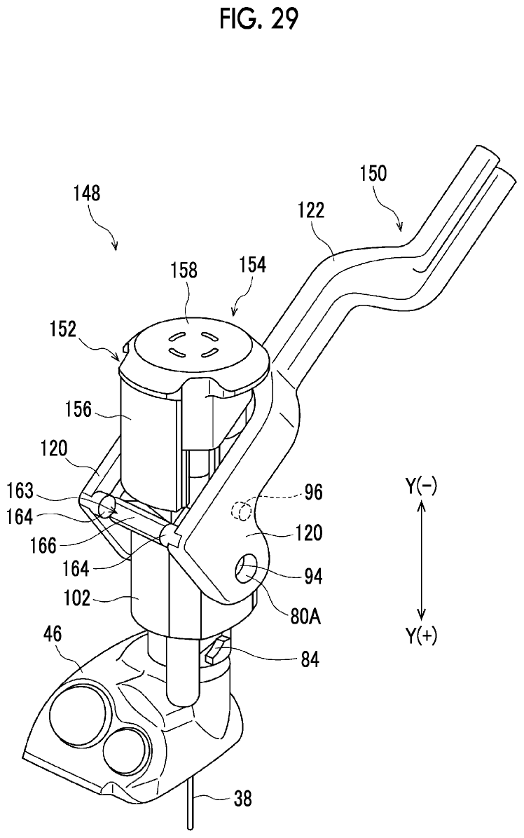

Next, a wire fixation mechanism 148 of a second embodiment will be described with reference to FIGS. 29 to 31.

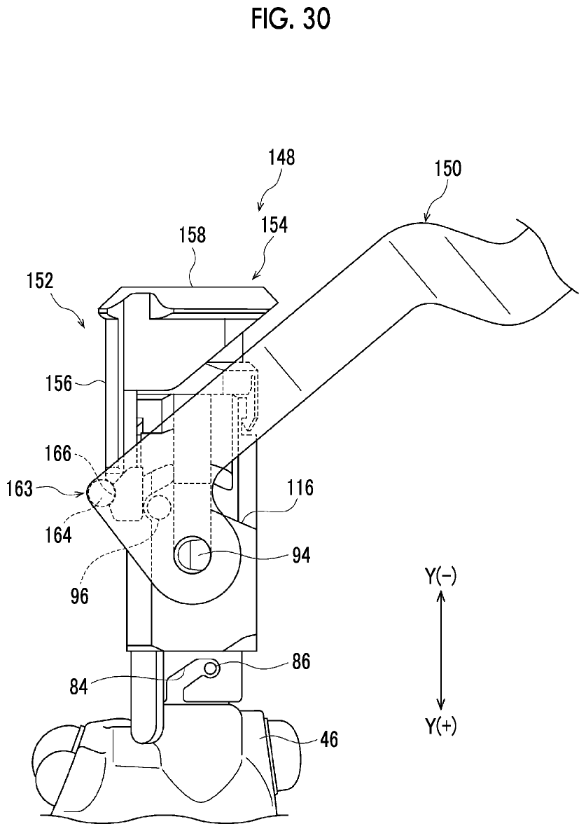

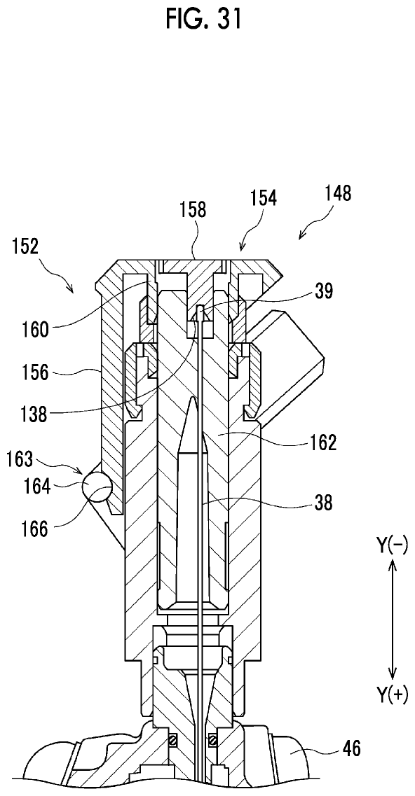

FIG. 29 is a perspective view showing a partially see-through view of the state of connection between a sliding lever 150 and a fixation unit 152 constituting the wire fixation mechanism 148. FIG. 30 is a front view of the wire fixation mechanism 148 shown in FIG. 29. FIG. 31 is a cross-sectional view of the wire fixation mechanism 148 shown in FIG. 30. All of FIG. 29 to FIG. 31 show a state at the end of the “rotational mounting operation”. Note that the same components as those in the first embodiment are given the same reference numerals and detailed description thereof may be omitted.

First, a difference between the first embodiment and the second embodiment will be described.

In the first embodiment, the fixing member 106 is moved to the fixing position as the “lever connection operation” of the sliding lever 80 is performed so that the locking target portion 39 is fixed by the fixing member 106. However, in the second embodiment, a fixing member 154 is manually moved to a fixing position so that the locking target portion 39 is fixed by the fixing member 154. The fixing member 154 functions as a fixation portion according to the embodiment of the present invention.

Hereinafter, the second embodiment will be described.

As shown in FIGS. 29 to 31, the wire fixation mechanism 148 includes the fixing member 154, and the fixing member 154 includes a substantially cylindrical main body portion 156 and a disk-shaped button portion 158. The main body portion 156 is mounted on an outer peripheral surface of the catch guide 102 to be movable along the Y-axis direction.

The button portion 158 is a portion that is pushed by a finger of the operator and as shown in FIG. 31, a cylindrical slide portion 160 protrudes to the Y (+) direction side from an inner surface of the button portion 158. The slide portion 160 is mounted to an end portion of a catch main body 162 that is on the Y (−) direction side such that the slide portion 160 is movable along the Y-axis direction. In addition, the fixation hole 138 that is engaged with the locking target portion 39 of the wire 38 is provided in an inner surface of the button portion 158. Note that, the locking hole 137 that has an opening shape in which the first hole 134 and the second hole 136 are continuously connected to each other is formed in the above-described catch main body 162 as well as with the catch main body 104 shown in FIG. 16.

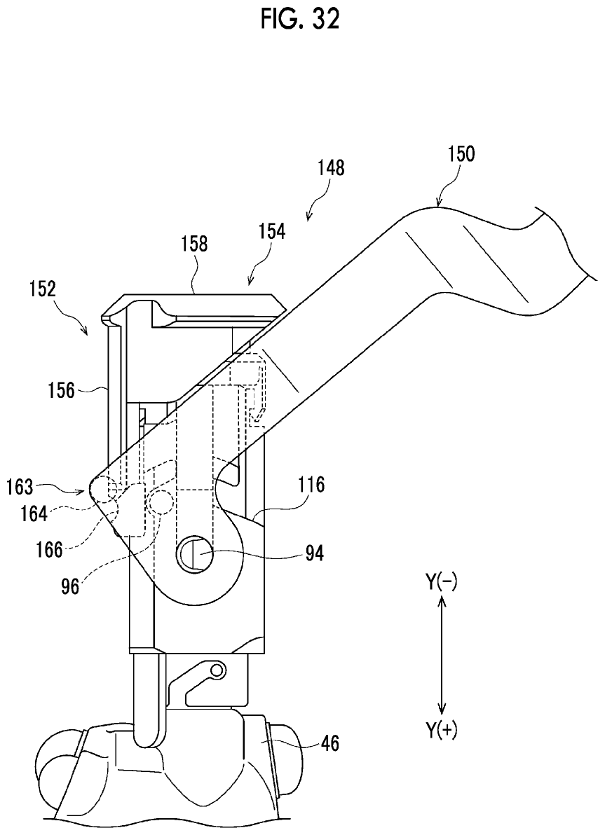

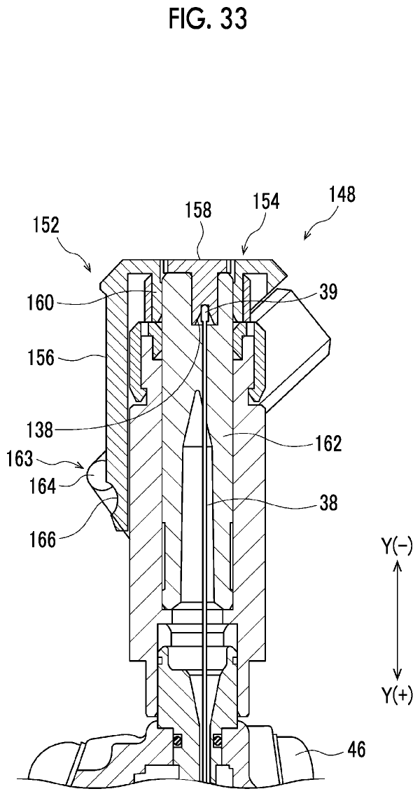

According to the wire fixation mechanism 148 configured as described above, in a case where the button portion 158 is manually pushed in the Y (+) direction as shown in FIGS. 32 and 33, the fixing member 154 is moved in the Y (+) direction by being guided by the slide portion 160. Because of the above-described operation, the locking target portion 39 is locked at the second hole 136 (refer to FIG. 19) and the locking target portion 39 is interposed between the fixation hole 138 and the catch main body 162. Accordingly, a locked state between the locking target portion 39 and the second hole 136 is fixed by the fixing member 154, and the wire 38 is reliably fixed to the wire fixation mechanism 148. Note that, for example, the fixing member 154 is held, because of frictional resistance between the slide portion 160 and the catch main body 162, at a position to which the fixing member 154 is moved as described above.

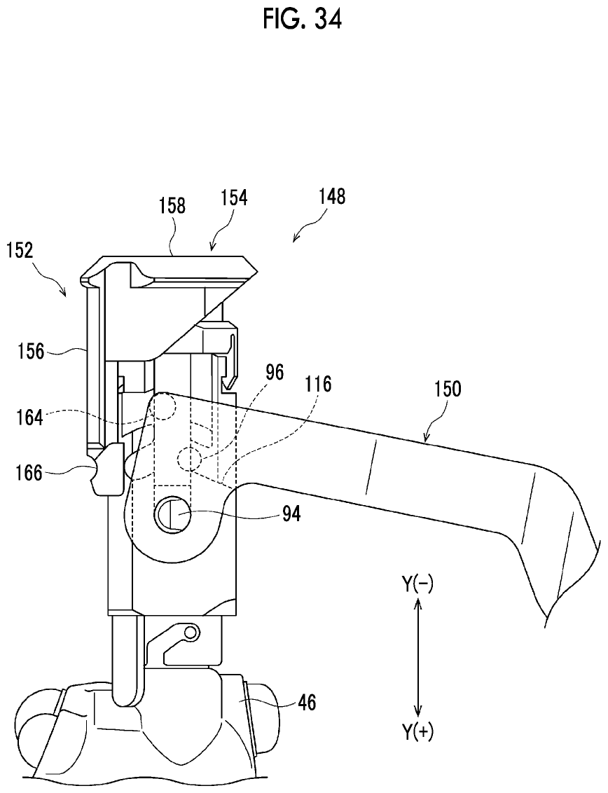

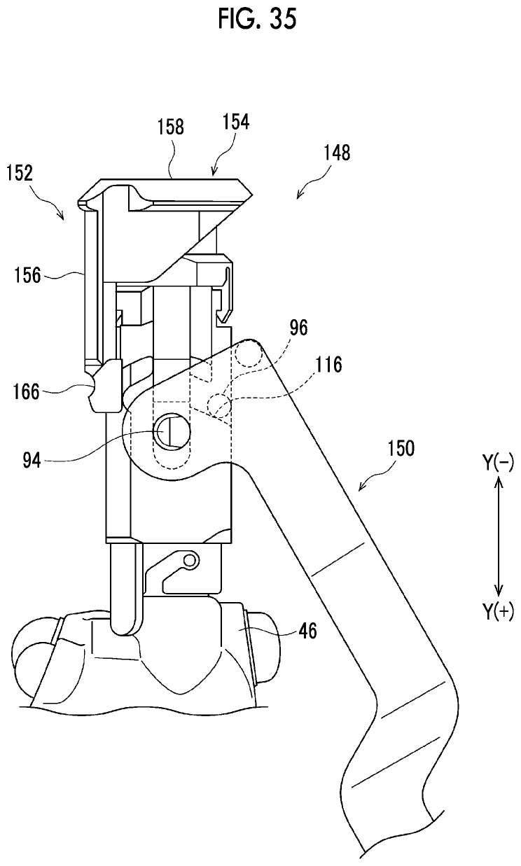

Thereafter, in a case where the “lever connection operation” is started, the sliding lever 150 rotates in a clockwise direction with the first shafts 94 as a rotation axis thereof as shown in FIG. 34. Then, as shown in FIG. 35, the second shafts 96 abut the second restriction surfaces 116, and the second restriction surfaces 116 restrict the movement of the sliding lever 150.

Then, in the second half of the “lever connection operation”, the rotation axis of the sliding lever 150 is switched from the first shafts 94 to the second shafts 96 with the second shafts 96 moving to the outside of the catch main body 162 along the second restriction surfaces 116. Then, the sliding lever 150 rotates with the second shafts 96 as a rotation axis, and the fixing member 154 and the catch main body 162 integrally move in the Y (−) direction so that wire 38 is pulled up. Then, the sliding lever 150 is connected to the link member 88 (refer to FIG. 10). Note that, since an operation of elevating and laying down the elevator 36 (refer to FIG. 2) that is performed thereafter is the same as that of the first embodiment, the description thereof will be omitted.

Meanwhile, in the second embodiment, in a case where the sliding lever 150 is connected to the link member 88 with the “lever connection operation” performed before an operation of manually pushing the button portion 158, an operation of elevating and laying down the elevator 36 (refer to FIG. 2) cannot be performed since the wire 38 has not been fixed to the wire fixation mechanism 148.

Therefore, the second embodiment has a configuration as follows so that the above-described problem is solved.

That is, the wire fixation mechanism 148 includes an operation restriction portion 163 that is selectively switchable between a restriction state in which the “lever connection operation” is restricted in a case where the fixing member 154 is at the unfixing position and an allowance state in which the “lever connection operation” is allowed in a case where the fixing member 154 is at the fixing position.

For example, as shown in FIG. 29, the operation restriction portion 163 includes columnar bosses 164 and 164 and an arc-shaped groove 166 that is engaged with the bosses 164 and 164. The bosses 164 protrude from inner surfaces of the plate-shaped portions 120 and 120 of the sliding lever 150 that face each other, the bosses 164 facing each other. In addition, the groove 166 is formed on an outer peripheral surface of the main body portion 156 of the fixing member 154.

The bosses 164 and the groove 166 are engaged with each other at the unfixing position shown in FIGS. 29 to 31 and at this time, the wire fixation mechanism 148 is held in the restriction state where the “lever connection operation” is restricted. In addition, in a case where the operation of manually pushing the button portion 158 is performed so that the fixing member 154 is moved to the fixing position with the button portion 158 as described above, the groove 166 is moved in the Y (+) direction as shown in FIGS. 32 and 33 and the groove 166 is disengaged from the bosses 164. Accordingly, the wire fixation mechanism 148 switches to the allowance state in which the “lever connection operation” is allowed, and the sliding lever 150 can be connected to the link member 88 by means of the “lever connection operation” performed thereafter.

As described above, in the second embodiment, the operation restriction portion 163 is provided. Therefore, the “lever connection operation” can be restricted in a case where the fixing member 154 is at the unfixing position and the “lever connection operation” can be allowed in a case where the fixing member 154 is at the fixing position. Accordingly, an operation of laying down the elevator 36 (refer to FIG. 2) by means of the elevation operation lever 20 can be reliably performed.

Hereinafter, embodiments of several wire fixation mechanisms will be described.

<Third Embodiment>

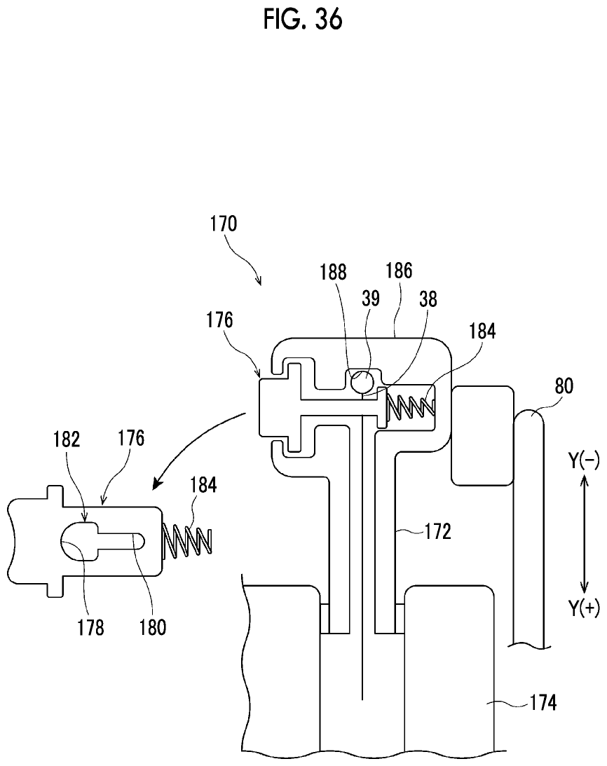

FIG. 36 is a cross-sectional view showing a main part of a wire fixation mechanism 170 according to a third embodiment. Note that the same components as those of the wire fixation mechanism 78 in the first embodiment are given the same reference numerals and detailed description thereof may be omitted.

The wire fixation mechanism 170 shown in FIG. 36 includes a wire catch 172, a catch guide 174, and the sliding lever 80. The wire catch 172 is connected to the sliding lever 80.

The wire catch 172 includes a fastener 176. The fastener 176 includes a locking hole 182 into which the locking target portion 39 is insertable and at which the locking target portion 39 is lockable. The locking hole 182 has an opening shape in which a semi-arc-shaped first hole 178 and a long second hole 180 are continuously connected to each other. The first hole 178 is formed to have an outer shape larger than that of the locking target portion 39 which has a spherical shape. Meanwhile, the second hole 180 is formed to have a width larger than a wire diameter but smaller than that of the locking target portion 39. The locking hole 182 functions as a locking hole according to the embodiment of the present invention.

In addition, the fastener 176 is movably attached to the wire catch 172 via a spring 184 in a direction orthogonal to the wire axis direction (the Y-axis direction). The fastener 176 functions as a locking member according to the embodiment of the present invention.

In addition, the wire catch 172 includes a fixation portion 186. The fixation portion 186 includes a fixation hole 188 with which the locking target portion 39 is engaged. The fixation hole 188 is formed at a position facing the second hole 180 in the Y-axis direction in FIG. 36.

According to the wire fixation mechanism 170 configured as described above, in a case where the wire 38 is accommodated in the wire catch 172, first, the fastener 176 is pushed into the wire catch 172 against a biasing force of the spring 184. Accordingly, the first hole 178 faces the locking target portion 39 and thus the locking target portion 39 passes through the first hole 178 and is engaged with the fixation hole 188. The position of the fastener 176 in this case corresponds to an insertion position.

Next, the fastener 176 is returned to a position shown in FIG. 36 by means of the biasing force of the spring 184. Accordingly, the second hole 180 faces the locking target portion 39. Therefore, the locking target portion 39 and the second hole 180 are locked and a locked state between the locking target portion 39 and the second hole 180 is fixed by the fixation portion 186. The position of the fastener 176 in this case corresponds to a locking position.

Therefore, according to an endoscope including the wire fixation mechanism 170 of the third embodiment, since the wire fixation mechanism 170 includes the wire catch 172, the catch guide 174, and the sliding lever 80, the wire 38 includes the locking target portion 39, and the wire catch 172 includes the fastener 176 in which the locking hole 182, into which the locking target portion 39 is insertable and at which the locking target portion 39 is lockable, is formed and the fixation portion 186 that fixes the locked state between the locking target portion 39 and the locking hole 182, the wire 38 can be reliably fixed to the wire fixation mechanism 170.

<Fourth Embodiment>

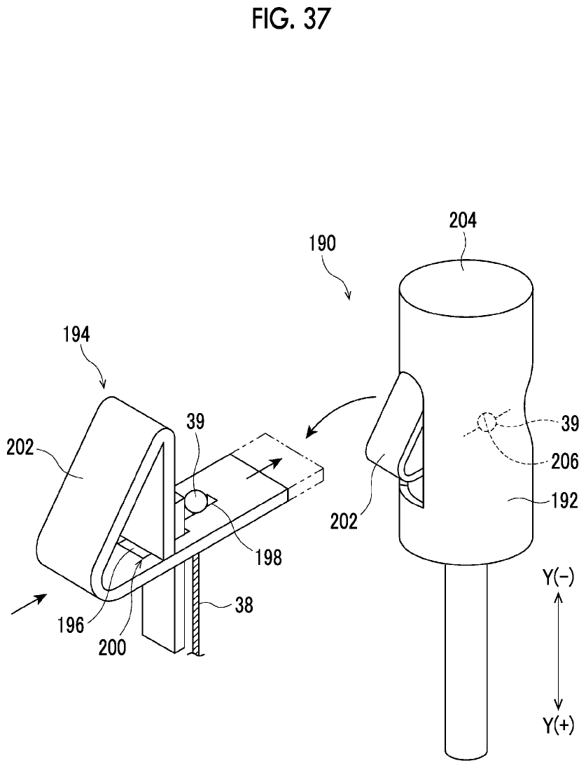

FIG. 37 is a perspective view of a wire fixation mechanism 190 according to a fourth embodiment. Note that the same components as those of the wire fixation mechanism 78 in the first embodiment are given the same reference numerals and detailed description thereof may be omitted.

The wire fixation mechanism 190 shown in FIG. 37 includes a wire catch 192. Note that, although not shown in FIG. 37, the wire fixation mechanism 190 includes a catch guide and a sliding lever.

The wire catch 192 includes a fastener 194. The fastener 194 includes a locking hole 200 into which the locking target portion 39 is insertable and at which the locking target portion 39 is lockable. The locking hole 200 has an opening shape in which a rectangular first hole 196 and a long second hole 198 formed to be narrower than the first hole 196 are continuously connected to each other. The first hole 196 is formed to have an outer shape larger than that of the locking target portion 39. Meanwhile, the second hole 198 is formed to have a width larger than the wire diameter but smaller than that of the locking target portion 39. The locking hole 200 functions as a locking hole according to the embodiment of the present invention.

In addition, the fastener 194 includes a leaf spring 202 bent and formed into a triangular shape. The fastener 194 is attached to the wire catch 192 via the leaf spring 202 in a direction orthogonal to the wire axis direction (the Y-axis direction). The fastener 194 functions as a locking member according to the embodiment of the present invention.

In addition, the wire catch 192 includes a fixation portion 204. The fixation portion 204 includes a fixation hole 206 with which the locking target portion 39 is engaged. The fixation hole 206 is formed at a position facing the second hole 198 in the Y-axis direction in FIG. 37.

According to the wire fixation mechanism 190 configured as described above, in a case where the wire 38 is accommodated in the wire catch 192, first, the fastener 194 is pushed into the wire catch 192 against a biasing force of the leaf spring 202. Accordingly, the first hole 196 faces the locking target portion 39 and thus the locking target portion 39 passes through the first hole 196 and is engaged with the fixation hole 206. The position of the fastener 194 in this case corresponds to the insertion position.

Next, the fastener 194 is returned to a position shown in FIG. 37 by means of the biasing force of the leaf spring 202. Accordingly, the second hole 198 faces the locking target portion 39. Therefore, the locking target portion 39 and the second hole 198 are locked and a locked state between the locking target portion 39 and the second hole 198 is fixed by the fixation portion 204. The position of the fastener 194 in this case corresponds to the locking position.

Therefore, even in the case of an endoscope including the wire fixation mechanism 190 of the fourth embodiment, the wire 38 can be reliably fixed to the wire fixation mechanism 190 as with the third embodiment.

<Fifth Embodiment>

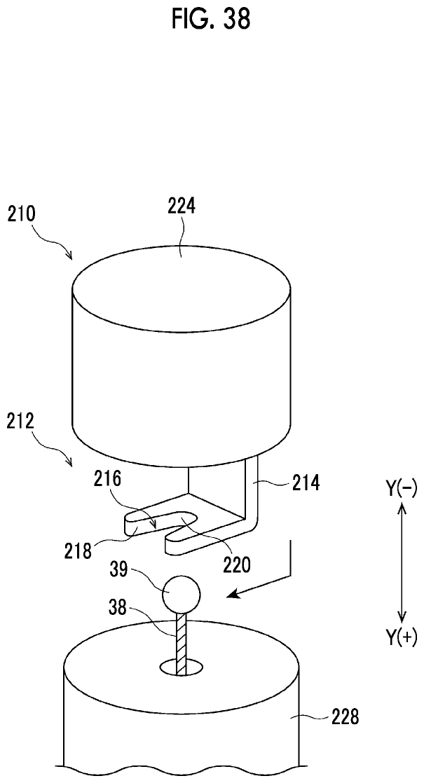

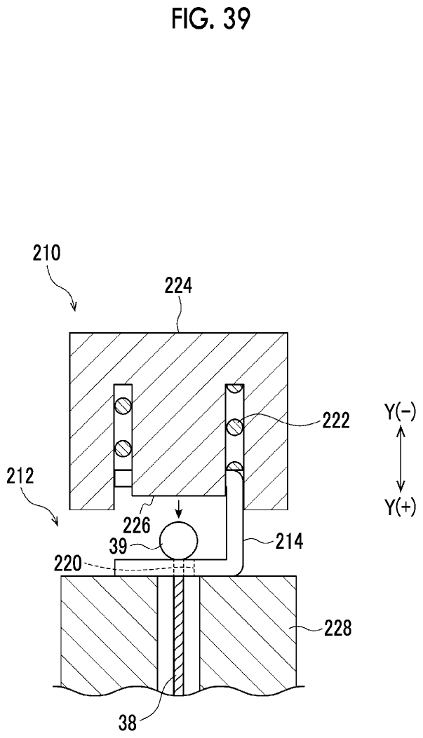

FIG. 38 is a perspective view of a wire fixation mechanism 210 according to a fifth embodiment, and FIG. 39 is a cross-sectional view of the wire fixation mechanism 210. Note that the same components as those of the wire fixation mechanism 78 in the first embodiment are given the same reference numerals and detailed description thereof may be omitted.

The wire fixation mechanism 210 shown in FIGS. 38 and 39 includes a wire catch 212. The wire catch 212 includes a catch main body 228 and a fixing member 224. The fixing member 224 functions as a fixation portion according to the embodiment of the present invention. Note that, although not shown in FIGS. 38 and 39, the wire fixation mechanism 210 includes a catch guide and a sliding lever.

The wire catch 212 includes an L-shaped fastener 214. A V-shaped groove 216 is formed in the fastener 214. The groove 216 includes a wide portion 218 on an opening side and a narrow portion 220 on a side opposite to the opening side. The wide portion 218 is formed to be larger than the outer shape of the locking target portion 39. Meanwhile, the narrow portion 220 is formed to have a width larger than the wire diameter but smaller than that of the locking target portion 39. The groove 216 functions as a locking hole according to the embodiment of the present invention.

In addition, the fastener 214 is attached to the fixing member 224 via a spring 222 that biases the fastener 214 in the Y (+) direction. The fastener 214 functions as a locking member according to the embodiment of the present invention.

The fixing member 224 includes an engagement surface 226 that is engaged with the locking target portion 39 of the wire 38. The engagement surface 226 is formed at a position facing the narrow portion 220 of the groove 216 in the Y-axis direction in FIG. 39. Note that although the engagement surface 226 is a flat surface in FIG. 39, the same hole shape as a fixation hole according to the embodiment of the present invention in which an opening portion, into which the locking target portion 39 is accommodable, is formed may also be adopted. In this case, it is preferable that at least a portion of an inner wall surface of the hole is formed as a restriction surface. The restriction surface is a surface that restricts the movement of the locking target portion 39 in a direction orthogonal to the wire axis direction.

According to the wire fixation mechanism 210 configured as described above, in a case where the wire 38 is accommodated into the catch main body 228, the locking target portion 39 abuts the narrow portion 220 of the groove 216 of the fastener 214. In a case where the above-described accommodation operation is continued thereafter, the fastener 214 is pushed by the locking target portion 39 and oscillates against a biasing force of the spring 222. Because of this operation, the wide portion 218 of the groove 216 faces the locking target portion 39, and the locking target portion 39 passes through the groove 216. In addition, immediately after the passage, the fastener 214 is returned to a position shown in FIG. 39 by the biasing force of the spring 222. Accordingly, the locking target portion 39 and the narrow portion 220 are locked. Thereafter, the fixing member 224 is fixed to the catch main body 228 by a fixing unit (not shown), so that the engagement surface 226 is engaged with the locking target portion 39. Accordingly, a locked state between the locking target portion 39 and the narrow portion 220 is fixed by the fixing member 224. Note that examples of the fixing unit include a fixing unit that uses a screw, a fixing unit that uses engagement such as snap fitting, and a fixing unit that uses press fitting.

Therefore, even in the case of an endoscope including the wire fixation mechanism 210 of the fifth embodiment, the wire 38 can be reliably fixed to the wire fixation mechanism 210.

<Sixth Embodiment>

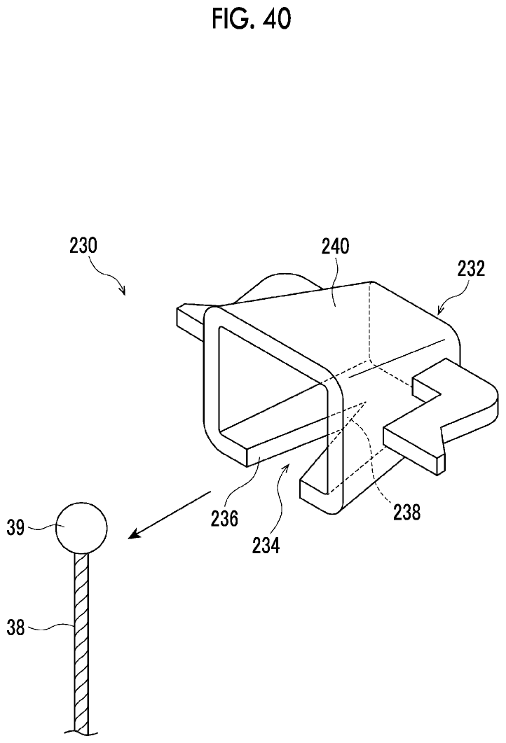

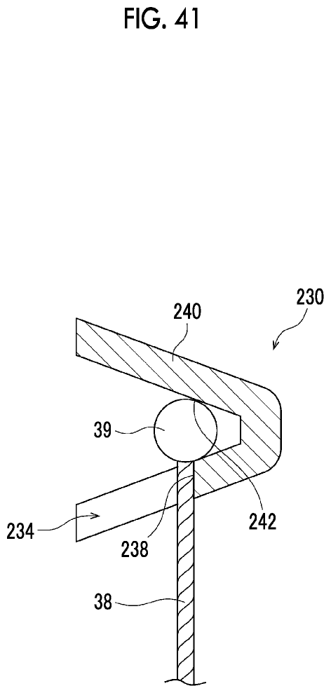

FIG. 40 is a perspective view of a wire fixation mechanism 230 according to a sixth embodiment, and FIG. 41 is a cross-sectional view of the wire fixation mechanism 230. Note that the same components as those of the wire fixation mechanism 78 in the first embodiment are given the same reference numerals and detailed description thereof may be omitted.

The wire fixation mechanism 230 shown in FIGS. 40 and 41 includes a wire catch 232. Note that, although not shown in FIGS. 40 and 41, the wire fixation mechanism 230 includes a catch guide and a sliding lever.

A V-shaped groove 234 is formed at the wire catch 232. The groove 234 includes a wide portion 236 on an opening side and a narrow portion 238 on a side opposite to the opening side. The wide portion 236 is formed to be larger than the outer shape of the locking target portion 39. Meanwhile, the narrow portion 238 is formed to have a width larger than the wire diameter but smaller than that of the locking target portion 39. The groove 234 functions as a locking hole according to the embodiment of the present invention.

The wire catch 232 includes a fixation portion 240. The fixation portion 240 includes an engagement surface 242 that is engaged with the locking target portion 39. The engagement surface 242 is formed at a position facing the narrow portion 238 in the Y-axis direction in FIG. 41.

According to the wire fixation mechanism 230 configured as described above, the wire catch 232 is inserted onto the wire 38 in a direction orthogonal to the wire axis direction (the Y-axis direction). As a result, the locking target portion 39 and the narrow portion 238 of the groove 234 are locked and the engagement surface 242 is engaged with the locking target portion 39. Accordingly, a locked state between the locking target portion 39 and the narrow portion 238 is fixed by the fixation portion 240.

Therefore, even in the case of an endoscope including the wire fixation mechanism 230 of the sixth embodiment, the wire 38 can be reliably fixed to the wire fixation mechanism 230.

<Seventh Embodiment>

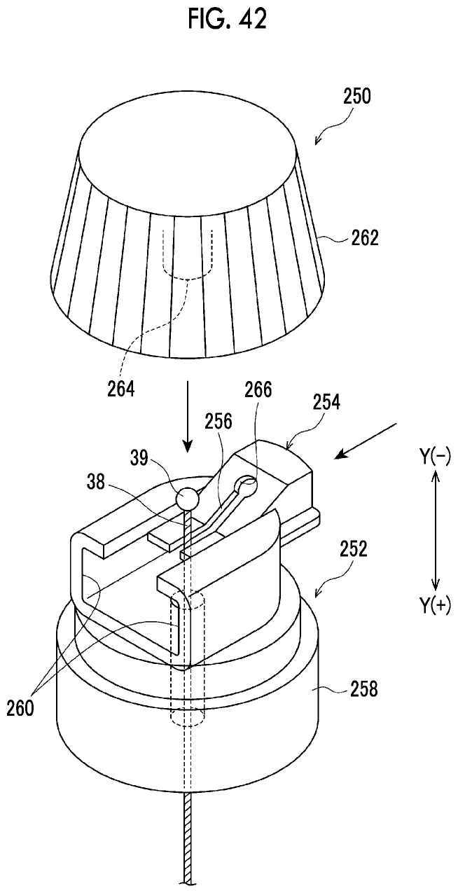

FIG. 42 is an assembly perspective view of a wire fixation mechanism 250 according to a seventh embodiment. Note that the same components as those of the wire fixation mechanism 78 in the first embodiment are given the same reference numerals and detailed description thereof may be omitted.

The wire fixation mechanism 250 shown in FIG. 42 includes a wire catch 252. The wire catch 252 includes a catch main body 258 and a fixing member 262. The fixing member 262 functions as a fixation portion according to the embodiment of the present invention. Note that, although not shown in FIG. 42, the wire fixation mechanism 250 includes a catch guide and a sliding lever.

The wire catch 252 includes a wedge-shaped fastener 254. In the fastener 254, a slit 256 is formed in a direction orthogonal to the wire axis direction (the Y-axis direction). The slit 256 is formed to have a width larger than the wire diameter but smaller than that of the locking target portion 39.

In the catch main body 258 of the wire catch 252, a guide groove 260 that guides the fastener 254 to be movable in a direction orthogonal to the wire axis direction is formed.

The fixing member 262 of the wire catch 252 is configured in a cap-like shape, and the fixing member 262 includes, as an inner surface thereof, an engagement surface 264 that is engaged with the locking target portion 39 of the wire 38. The engagement surface 264 is formed at a position facing a terminal end portion 266 of the slit 256 in the Y-axis direction in FIG. 42.

According to the wire fixation mechanism 250 configured as described above, the fastener 254 is inserted onto, in a direction orthogonal to the wire axis direction (the Y-axis direction), the wire 38 accommodated in the catch main body 258. Accordingly, the locking target portion 39 and the terminal end portion 266 of the slit 256 are locked. Thereafter, the fixing member 262 is fixed to the catch main body 258 so that the engagement surface 264 abuts the locking target portion 39. Accordingly, a locked state between the locking target portion 39 and the terminal end portion 266 is fixed by the fixing member 262.

Therefore, even in the case of an endoscope including the wire fixation mechanism 250 of the seventh embodiment, the wire 38 can be reliably fixed to the wire fixation mechanism 250.

<Eighth Embodiment>

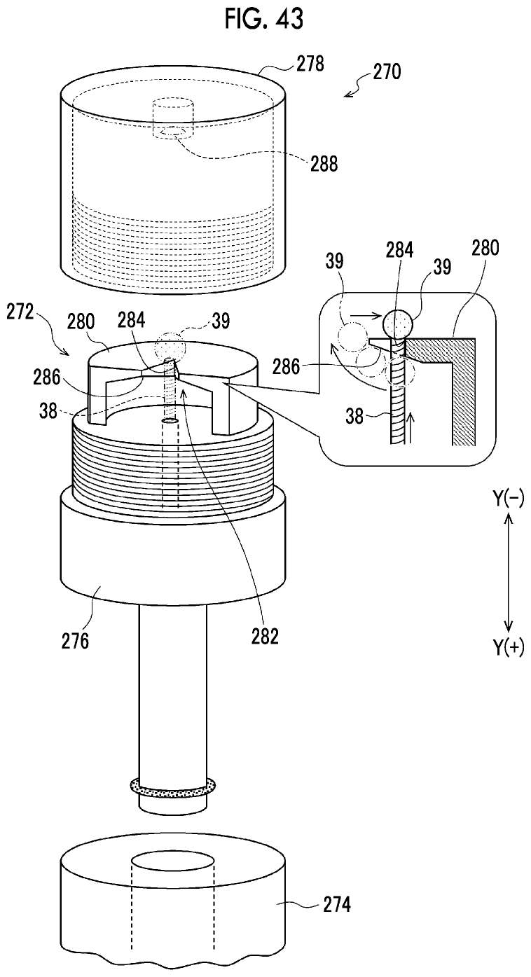

FIG. 43 is an assembly perspective view of a wire fixation mechanism 270 according to an eighth embodiment. Note that the same components as those of the wire fixation mechanism 78 in the first embodiment are given the same reference numerals and detailed description thereof may be omitted.

The wire fixation mechanism 270 shown in FIG. 43 includes a wire catch 272 and a catch guide 274. In addition, the wire catch 272 includes a catch main body 276 and a fixing member 278. The fixing member 278 functions as a fixation portion according to the embodiment of the present invention. Note that, although not shown in FIG. 43, the wire fixation mechanism 270 includes a sliding lever.

The catch main body 276 of the wire catch 272 includes a fastener 280. In the fastener 280, a V-shaped groove 282 is formed in a direction orthogonal to the wire axis direction (the Y-axis direction). A narrow portion 284 of the groove 282 is formed to have a width larger than the wire diameter but smaller than that of the locking target portion 39. The groove 282 functions as a locking hole according to the embodiment of the present invention.

In addition, on the fastener 280, a tapered surface 286 that abuts the locking target portion 39 and guides the locking target portion 39 toward the narrow portion 284 is formed.

In addition, the fixing member 278 of the wire catch 272 is configured in a cap-like shape open to the catch main body 276 side, and the fixing member 278 includes, as an inner surface thereof, an engagement surface 288 that is engaged with the locking target portion 39 of the wire 38. The engagement surface 288 is formed at a position facing the narrow portion 284 of the groove 282 in the Y-axis direction in FIG. 43.

According to the wire fixation mechanism 270 configured as described above, the locking target portion 39 of the wire 38 accommodated in the catch main body 276 abuts the tapered surface 286 of the fastener 280, is moved in the Y (−) direction while being guided by the tapered surface 286, and is locked at the narrow portion 284 of the groove 282. Thereafter, the fixing member 278 is fixed to the catch main body 276 by means of a screw so that the engagement surface 288 is engaged with the locking target portion 39. Accordingly, a locked state between the locking target portion 39 and the narrow portion 284 is fixed by the fixing member 278.

Therefore, even in the case of an endoscope including the wire fixation mechanism 270 of the eighth embodiment, the wire 38 can be reliably fixed to the wire fixation mechanism 270.

<Ninth Embodiment>

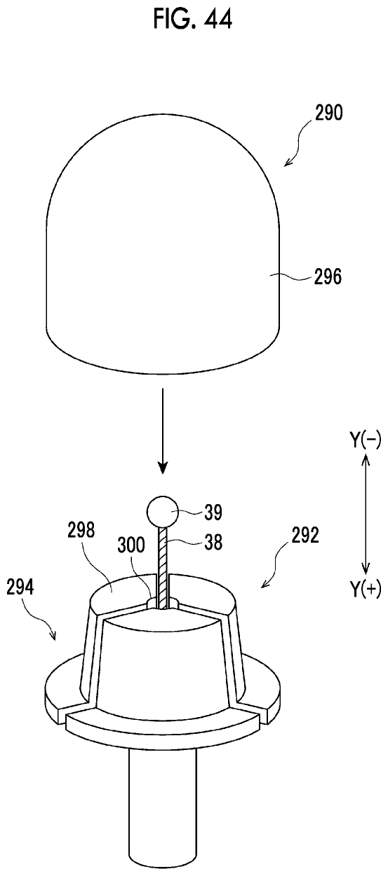

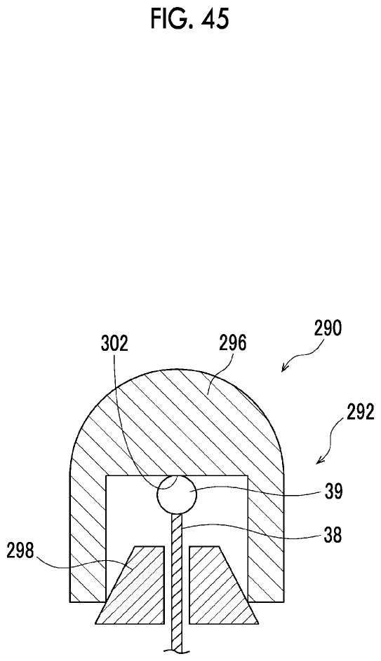

FIG. 44 is an assembly perspective view of a wire fixation mechanism 290 according to a ninth embodiment, and FIG. 45 is a cross-sectional view of FIG. 44. Note that the same components as those of the wire fixation mechanism 78 in the first embodiment are given the same reference numerals and detailed description thereof may be omitted.

The wire fixation mechanism 290 shown in FIGS. 44 and 45 includes a wire catch 292. In addition, the wire catch 292 includes a catch main body 294 and a fixing member 296. Note that although not shown in FIG. 44 and FIG. 45, the wire fixation mechanism 290 includes a catch guide and a sliding lever.

The catch main body 294 of the wire catch 292 includes a collet chuck 298. The collet chuck 298 includes an opening portion 300 into which the wire 38 is inserted, and the opening portion 300 is open such that the diameter thereof is larger than the diameter of the locking target portion 39 in a normal state. In a case where the cap-shaped fixing member 296 is mounted onto the catch main body 294, the diameter of the opening portion 300 is decreased to be smaller than the diameter of the locking target portion 39. The opening portion 300 functions as a locking hole according to the embodiment of the present invention.

The fixing member 296 includes, as an inner surface thereof, an engagement surface 302 that is engaged with the locking target portion 39. The engagement surface 302 is formed at a position facing the opening portion 300 in the Y-axis direction in FIG. 45.

According to the wire fixation mechanism 290 configured as described above, the locking target portion 39 of the wire 38 accommodated in the catch main body 294 passes through the opening portion 300 of the collet chuck 298 and protrudes in the Y (−) direction. Thereafter, the fixing member 296 is mounted onto the catch main body 294. Accordingly, the diameter of the opening portion 300 is decreased, so that the locking target portion 39 and the opening portion 300 are locked. Then, the engagement surface 302 is engaged with the locking target portion 39 and thus a locked state between the locking target portion 39 and the opening portion 300 is fixed by the fixing member 296.

Therefore, even in the case of an endoscope including the wire fixation mechanism 290 of the ninth embodiment, the wire 38 can be reliably fixed to the wire fixation mechanism 290. In addition, according to the wire fixation mechanism 290, with mounting surfaces between the catch main body 294 and the fixing member 296 being formed as tapered surfaces, it is possible to perform the above-described locking and fixation while decreasing the diameter of the opening portion 300.

Although an example in which the endoscope according to the embodiment of the present invention is applied to a duodenoscope has been described above, the technique of the present invention can also be applied to other endoscopes such as a colonoscope and a small intestinal endoscopy instead of being applied to a duodenoscope. In addition, regarding the present invention, some improvements or modifications may be made without departing from the gist of the present invention.

### EXPLANATION OF REFERENCES

10: endoscope
12: endoscope system
14: endoscope processor device
15: light source device
15A: processor-side connector
16: image processing device
18: display
20: elevation operation lever
22: hand-side operation portion
23: outlet port
25: connection portion
25A: axis
24: insertion portion
26: soft portion
28: bendable portion
30: distal end portion
32: distal end portion main body
34: cap
34A: opening window
34B: wall portion
34C: bearing
36: treatment tool elevator (elevator)
36A: treatment tool guide surface
36B: rotary shaft
37: treatment tool channel
38: elevation operation wire (wire)
39: locking target portion
40: wire channel
42: air/water supply tube
44: cable insertion channel
45: insertion channel
46: operation portion main body
46A: proximal end surface
48: grip portion
50: bend-proof tube
52: universal cable
54: connector device
57: air/water supply button
58: air/water supply nozzle
59: suction button
60: treatment tool outlet port
61: through-hole
62: angle knob
64: treatment tool inlet port
68: partition wall
68A: upper surface
74: illumination window
76: observation window
78: wire fixation mechanism
80: sliding lever
80A: lever bearing hole
82: fixation unit
84: cam groove
84A: opening end
86: cam pin
88: link member
90: opening portion
92: claw portion
94: first axis
96: second axis
98: unlocking member
100: wire catch
102: catch guide
102A: surface
103: boss hole
104: catch main body
105: first restriction surface
106: fixing member
106A: end surface
107: cap
108: connection portion
110: claw portion
112: groove
114: catch guide groove
116: second restriction surface
120: plate-shaped portion
121: boss
122: lever main body
124: cam groove
124A: right end
124B: left end
125A: first cam groove portion
125B: second cam groove portion
126: pin
130: columnar portion
132: guide portion
133: catch main body groove
134: first hole
135: opening portion
136: second hole
137: locking hole
138: fixation hole
138A: bottom portion
139: guide surface
148: wire fixation mechanism
150: sliding lever
152: fixation unit
154: fixing member
156: main body portion
158: button portion
160: slide portion
162: catch main body
163: operation restriction portion
164: boss
166: groove
170: wire fixation mechanism
172: wire catch
174: catch guide
176: fastener
178: first hole
180: second hole
182: locking hole
184: spring
186: fixation portion
188: fixation hole
190: wire fixation mechanism
192: wire catch
194: fastener
196: first hole
198: second hole
200: locking hole
202: leaf spring
204: fixation portion
206: fixation hole
210: wire fixation mechanism
212: wire catch
214: fastener
216: groove
218: wide portion
220: narrow portion
222: spring
224: fixing member
226: engagement surface
230: wire fixation mechanism
232: wire catch
234: groove
236: wide portion
238: narrow portion
240: fixation portion
242: engagement surface
250: wire fixation mechanism
252: wire catch
254: fastener
256: slit
258: catch main body
260: guide groove
262: fixing member
264: engagement surface
266: terminal end portion
270: wire fixation mechanism
272: wire catch
274: catch guide
276: catch main body
278: fixing member
280: fastener
282: groove
284: narrow portion
286: tapered surface
288: engagement surface
290: wire fixation mechanism
292: wire catch
294: catch main body
296: fixing member
298: collet chuck
300: opening portion
302: engagement surface

What is claimed is:

1. An endoscope comprising:
an operation portion that is provided with an operation member;
an insertion portion that is provided on a distal end side of the operation portion and that is inserted into a subject;
a treatment tool elevator that is provided at a distal end portion of the insertion portion;
an elevation operation wire of which a distal end side is connected to the treatment tool elevator and that is pushed and pulled, as the operation member is operated, so that the treatment tool elevator is operated; and
a wire fixation mechanism that fixes a proximal end side of the elevation operation wire,
wherein the wire fixation mechanism includes
a wire catch that attachably and detachably locks and fixes the proximal end side of the elevation operation wire,
a catch guide that guides the wire catch in a wire axis direction of the elevation operation wire, and
a sliding lever that operates as the operation member is operated so that the wire catch is moved forward and backward in the wire axis direction,
the elevation operation wire includes a locking target portion that is positioned on a proximal end side of a long wire main body and is formed to have an outer shape larger than that of the wire main body, and
the wire catch includes
a locking hole into which the locking target portion is insertable and at which the locking target portion is lockable, and
a fixation portion that fixes a locked state between the locking target portion inserted into the locking hole and the locking hole.

2. The endoscope according to claim 1,
wherein the fixation portion includes a restriction surface that restricts the locking target portion locked at the locking hole from moving in a direction orthogonal to the wire axis direction.

3. The endoscope according to claim 2,
wherein the fixation portion includes a fixation hole in which an opening portion, in which the locking target portion is accommodable, is formed and at least a portion of an inner wall surface of the fixation hole is configured as the restriction surface.

4. The endoscope according to claim 3,
wherein the fixation hole includes a conical guide surface that becomes narrower toward an inner portion of the fixation hole.

5. The endoscope according to claim 1,
wherein the locking hole has an opening shape in which a first hole of which a size is enough for the locking target portion to be inserted thereinto and a second hole of which an outer shape is larger than an outer shape of the wire main body and is smaller than an outer shape of the locking target portion are continuously connected to each other.

6. The endoscope according to claim 5,
wherein the wire catch is configured to be rotatable around a rotation axis eccentric from the elevation operation wire, and
the locking hole is provided at a position eccentric from the rotation axis and the first hole and the second hole are formed to be continuously connected to each other along a trajectory of rotation around the rotation axis.

7. The endoscope according to claim 1,
wherein the fixation portion is movable between a fixing position at which the locked state of the locking target portion and the locking hole is fixed and an unfixing position at which the locked state of the locking target portion and the locking hole is unfixed.

8. The endoscope according to claim 7,
wherein the operation portion includes a link member that is operated as the operation member is operated,
the sliding lever includes a lever connecting portion that is attachably and detachably connectable to the link member, and
the fixation portion is movable between the fixing position and the unfixing position as a lever connection operation of connecting the lever connecting portion to the link member is performed.

9. The endoscope according to claim 7,
wherein the operation portion includes a link member that is operated as the operation member is operated,
the sliding lever includes a lever connecting portion that is attachably and detachably connectable to the link member, and
the wire fixation mechanism includes an operation restriction portion that is selectively switchable between a restriction state in which a lever connection operation of connecting the lever connecting portion to the link member is restricted in a case where the fixation portion is at the unfixing position and an allowance state in which the lever connection operation is allowed in a case where the fixation portion is at the fixing position.

10. The endoscope according to claim 1,
wherein the wire catch includes a locking member that includes a locking hole, and
the locking member is movable between an insertion position at which the locking target portion is insertable into the locking hole and a locking position at which the locking target portion is locked at the locking hole.
